(19) United States
(12) Patent Application Publication
(10) Pub. No.: US 2026/0309711 A1
(43) Pub. Date: Oct. 8, 2026

(54) IDENTIFYING ANTIBODY VIRUS-NEUTRALIZATION STATUS

(71) Applicant: MASSACHUSETTS INSTITUTE OF TECHNOLOGY, Cambridge, MA (US)

(72) Inventors: Laura Kiessling, Cambridge, MA (US); Michael Wuo, Somerville, MA (US); Amanda Dugan, Arlington, MA (US)

(21) Appl. No.: 19/482,201

(22) PCT Filed: May 7, 2024
(86) PCT No.: PCT/US2024/028102

§ 371 Date: Nov. 6, 2025

Related U.S. Application Data
(60) Provisional application No. 63/500,610, filed on May 7, 2023.

(51) Int. Cl. G01N 33/569

(52) U.S. Cl. CPC G01N 33/56983; G01N 2333/165; G01N 2333/4724; G01N 2500/04

(57) ABSTRACT

In certain aspects, the invention provides methods and compositions for use in identifying a neutralizing status of a candidate antiviral agent. The invention also provides methods of determining one or more binding characteristics of glycan-binding agents.

### RELATED APPLICATIONS

This application claims benefit under 35 U.S.C. § 119(e) of U.S. Provisional application Ser. No. 63/500,610 filed May 7, 2023, the disclosure of which is incorporated by reference herein in its entirety.

### GOVERNMENT SUPPORT

This invention was made with government support under 5U01CA23107 awarded by the National Cancer Institute; R01 A1055258 awarded by the National Institute for Allergy and Infectious Disease; and F32 GM13311 awarded by the National Institute for General Medical Science. The government has certain rights in the invention.

### FIELD OF THE INVENTION

The invention relates, in part, to novel compositions and methods for identifying a virus-neutralization status of an anti-virus antibody.

### BACKGROUND

Viruses are the primary cause of respiratory tract infections worldwide, accounting for more than 2 million deaths annually (GBD 2016 Lower Respiratory Infections Collaborators, Lancet Infect. Dis. 2018, 18 (11), 1191-1210). Viral pathogens have been responsible for some of the most significant infectious disease outbreaks recorded (poliovirus, 1918 influenza A, 1957 Influenza A; HIV/AIDS; SARS-CoV-2). Many viral pathogens share a common strategy of employing densely glycosylated surface proteins to facilitate viral entry into host cells and modulate host immune defenses (Cook, J. D. and Lee, J. E., PLoS Pathog. 2013, 9 (5)). Because of their abundance and accessibility, viral glycoproteins often serve as key antigens upon which the host immune system mounts an attack. As the virus evolves, acquired mutations within these glycoproteins, such as the addition (Nabel, K. G.; et al., Science 2022, 375 (6578), eab16251) or remodeling of glycans (Casalino, L. et al., ACS Cent. Sci. 2020; Grant, O. C. et al., Sci. Rep. 2020, 10 (1), 14991), help the virus evade immune detection. Viral genome sequencing readily reveals amino acid mutations in emerging viral strains, yet alterations to protein glycosylation cannot be predicted from sequencing information alone. New tools to rapidly profile changes in viral protein glycosylation and their impact on immune recognition would be valuable assets in monitoring viral pathogens (Dugan, A. E. et al., Nat. Methods 2022, 19 (4), 384-387).

Human coronaviruses are emblematic of viruses that undergo mutation but the corresponding changes in protein glycosylation tend to be enigmatic. The coronavirus SARS-CoV-2 uses its Spike glycoprotein, which harbors significant glycosite microheterogeneity (V'kovski, P. et al., Nat. Rev. Microbiol. 2020, 1-16; Shajahan, A. et al., Glycobiology 2020 30.12: 981-988; Watanabe, Y. et al., Science 2020, eabb9983) (FIG. 1A), to mediate contacts with host receptors and facilitate entry into cells (Li, F., Annu. Rev. Virol. 2016, 3 (1), 237-261). Spike glycoprotein is a multidomain, class I fusion protein that exists as a homotrimer on the virion surface; the Spike protein is densely glycosylated, containing two putative O-glycosylation and 22 predicted N-glycosylation sites per monomer in the wild type (Wuhan-Hu-1) strain (FIG. 1B) (Shajahan, A. et al., Glycobiology 2020 30.12: 981-988; Watanabe, Y. et al., Science 2020, eabb9983). Although viral entry is primarily mediated by interactions between the receptor binding domain (RBD) of the Spike glycoprotein and host angiotensin-converting enzyme 2 (ACE2) receptor, recent studies have demonstrated Spike glycans enhance viral entry through interactions with host lectins (Lempp, F. A. et al., Nature 2021, 598 (7880), 342-347; Yang, Q. et al., eLife 2020, 9, e61552).

Changes in viral glycosylation pose a risk to global vaccination efforts. With SARS-C0V-2, the host humoral response is predominantly directed against the Spike protein (Grant, O. C. et al., Sci. Rep. 2020, 10 (1), 14991; Watanabe, Y. et al., Nat. Commun. 2020, 11 (1), 2688; Liu, L. et al., Nature 2020, 584 (7821), 450-456); still, not all antibodies that bind Spike can neutralize the virus and prevent viral entry into host cells (Brouwer, P. J. M. et al., Science 2020, 369 (6504), 643-650; Barnes, C. O. et al., Cell 2020, 182 (4), 828-842.e16). Moreover, variants with changes proximal to Spike glycosites have emerged during the pandemic (Xie, X. et al., Nat. Med. 2021, 27, 620-621; Wang, Y. et al., Nat. Commun. 2022, 13 (1), 871; S. Roberts, D. et al., Chem. Sci. 2022, 13 (36), 10944-10949). These evolved strains are of growing concern as they exhibit heightened transmissibility and may reduce the neutralization efficacy of convalescent or vaccinated patient antibodies (Nabel, K. G.; et al., Science 2022, 375 (6578), eab16251; Garcia-Beltran, W. F. et al., Cell 2021, 184 (9), 2372-2383.e9; Dejnirattisai, W. et al., Cell 2021, 184 (11), 2939-2954.e9; Harvey, W. T. et al., Nat. Rev. Microbiol. 2021, 19 (7), 409-424).

Two types of assays are traditionally used to determine antibody neutralization capacity of SARS CoV-2: 1) cellular assays that measure the ability of antibodies to block viral infection; or 2) in vitro assays that screen for the ability of antibodies to directly compete with Ace2 receptor binding. The former requires specialized equipment/facilities and is low throughput, and the latter assay does not account for antibodies that bind Spike at positions distal to the Ace2 recognition site but are nonetheless capable of neutralizing the virus. As such, complementary platforms that rapidly screen patient antibodies for Spike-neutralizing or non-neutralizing capabilities would be advantageous for understanding the spread of SARS-CoV-2 and informing therapeutic and vaccine design.

### SUMMARY OF ASPECTS OF THE INVENTION

According to an aspect of the invention, a method of identifying a virus-neutralization status of a candidate antiviral agent is provided, the method including: (a) contacting a virion of a virus that has previously contracted an anti-virus antibody, with one or more preselected lectin molecules, wherein the contacting results in a virion/lectin molecule complex; (b) contacting the virion/lectin molecule complex with a candidate antiviral agent that is a candidate to block infection with the virus; and (c) determining one or more binding characteristics of the preselected lectin molecule(s) with the antibody-contacted virion subsequent to the contacting with the candidate antiviral agent, wherein the determined one or more binding characteristics identifies the virus-neutralization status of the antiviral agent. In some embodiments, the one or more binding characteristics are determined for binding of the one or more preselected lectin molecules with a virion protein. In certain embodiments, the candidate agent to block infection with the virus is an anti-virus antibody. In certain embodiments, the candidate agent to neutralize the virus is a peptide binder, a protein, a small-molecule candidate agent. In some embodiments, neutralizing the virus includes blocking infection of the subject by the virus. In some embodiments, neutralizing the virus includes blocking entry of the viral particle of the virus into a cell. In certain embodiments, the determined binding characteristics include one or more of (a) the presence or absence of binding of the one or more preselected lectin molecule(s) with the virion protein; (b) a level of binding of the one or more preselected lectin molecules with the virion protein; (c) one or more location of the binding of the one or more preselected lectin molecules on the virion protein; (d) identity of the one or more preselected lectin molecules that bind the virion protein; and (e) identity of the one or more preselected lectin molecules that do not bind the virion protein. In certain embodiments, the determined binding characteristics include one or more of (a) the presence or absence of binding of the one or more preselected lectin molecule(s) with the virion protein; (b) a level of binding of the one or more preselected lectin molecules with the virion protein; (c) one or more location of the binding of the one or more preselected lectin molecules on the virion protein; (d) identity of the one or more preselected lectin molecules that bind the virion protein; and (e) identity of the one or more preselected lectin molecules that do not bind the virion protein. In some embodiments, the method also includes comparing binding characteristics determined with the method with control binding characteristics to identify the virus-neutralization status of the antiviral agent. In certain embodiments, the control binding characteristics include binding characteristics of the lectins in the virion/lectin molecule complex prior to contact with the candidate antiviral agent. In certain embodiments, the anti-viral antibody is obtained from a first subject known to have had been infected by the virus. In some embodiments, the anti-viral antibody is obtained from convalescent serum obtained from the first subject. In some embodiments, the anti-viral antibody is obtained from a first subject known to not have been infected by the virus but to have received one or more vaccinations against the virus. In some embodiments, the anti-virus antibody is a manufactured antibody. In certain embodiments, the anti-virus antibody is a recombinant antibody. In certain embodiments, the anti-virus antibody is a purified antibody. In some embodiments, the virion protein is a spike protein of the virion. In some embodiments, the spike protein is a full-length spike (FL-Spike) protein. In certain embodiments, the one or more preselected lectin molecules binds to the spike protein in the receptor-binding domain (RBD) of the spike protein. In some embodiments, the virus is an RNA virus. In some embodiments, the virus is a positive single-stranded RNA virus, and optionally is a member of the viral family Astroviridae, Caliciviridae Picornaviridae, Coronaviridae, Flaviviridae, Retroviridae, or Togaviridae. In certain embodiments, the virus is a MERS-CoV virus or a SARS-CoV virus. In certain embodiments, virus is a SARS-CoV-1, SARS-CoV-2 virus, or an infective functional variant thereof. In some embodiments, the virus is a negative single-stranded RNA virus, and optionally is a member of the viral family Arenaviridae, Bunyaviridae, Filoviridae, Orthomyxoviridae, Paramyxoviridae, or Rhabdoviridae. In some embodiments, the virus is an enveloped virus. In certain embodiments, the one or more preselected lectin molecules include one or more eukaryotic lectins, prokaryotic lectins, and plant lectins. In certain embodiments, the one or more preselected lectin molecules include one or more mammalian lectins. In some embodiments, a method of determining the one or more binding characteristics includes an enzyme-linked lectin assay (ELLA). In some embodiments, determining the one or more binding characteristics includes a high-throughput method. In some embodiments, one or more of the preselected lectin molecules is detectably labeled. In certain embodiments, one or more of the preselected glycan-binding agents is detectably labeled. In some embodiments, the detectable label includes biotin. In some embodiments, the one or more preselected lectin molecule(s) contacting the virion include one or more of: Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-aGal, RPL-Gal1, RPL-aMan, RPL-Man2, RPL-Sia1, and RPL-Fuc1. In certain embodiments, the one or more preselected lectin molecules contacting the virion include 1, 2, 3, 4, 5, 6, 7, 8, 9, 10, 11, 12, 13, 14, 15, 16, 17, 18, 19, 20, 21, 22, 23, 24, 25, 26, 27, 28 or 29 of Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1. In certain embodiments, the virus is a SARS-CoV-2 virus, the anti-virus antibody is a purified antibody, and the one or more preselected lectin molecules contacting the virion protein comprise: MALI, RPL-αMan, RPL-Man2, PNA, RPL-Gal1, Jacalin, RPL-αGal, RPL-Sia1, RPL-Fuc1, DBA, and LTL. In some embodiments, the one or more preselected lectin molecules contacting the virion include Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1. In some embodiments, the virus is a SARS-CoV-2 virus, the anti-virus antibody is a purified antibody, and the one or more preselected lectin molecules contacting the virion protein comprise: MALI, RPL-αMan, RPL-Man2, PNA, RPL-Gal1, Jacalin, RPL-αGal, RPL-Sia1, RPL-Fuc1, DBA, and LTL. In certain embodiments, determining the presence of binding of the one or more preselected lectin molecules MALI, RPL-αMan, RPL-Man2, PNA, RPL-Gal1, Jacalin, RPL-αGal, RPL-Sia1, RPL-Fuc1, DBA, and LTL to the virion protein identifies the anti-virus antibody as a SARS-CoV-2-neutralizing antibody. In some embodiments, the virus is a SARS-CoV-2 virus, the anti-viral antibody is obtained from a subject serum sample, and the one or more preselected lectin molecules contacting the virion comprise: ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1. In certain embodiments, the subject serum sample is a convalescent serum sample. In some embodiments, the subject serum sample is obtained from a subject that has not been infected with the virus. In some embodiments, the serum sample is obtained from a subject that has not been infected with the virus but has been vaccinated one or more times against the virus. In certain embodiments, the serum sample is obtained from a subject that has been infected with the virus and has been vaccinated one or more times against the virus. In certain embodiments, determining the presence of binding of the one or more preselected lectin molecules ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1 to the virion protein identifies the anti-virus antibody as a SARS-CoV-2-neutralizing antibody. In some embodiments, the method also includes administering the anti-viral antibody identified as neutralizing the virus to a second subject as a treatment for a SARS-CoV-2 viral infection in the second subject.

According to another aspect of the invention, a method of identifying a candidate antibody to treat an infection by a SARS-CoV-2 virus is provided, the method including: (a) determining whether an anti-virus antibody has a status of neutralizing the virus using a method set forth in any embodiment of a method of an aforementioned aspect of the invention. (b) further assessing the anti-virus antibody determined to have the status of neutralizing the virus for efficacy and safety; wherein the further assessment includes: (i) contacting a plurality of cells infected with the SARS-CoV-2 virus with the anti-viral antibody determined to have the neutralizing status; (ii) administering the anti-viral antibody determined to have the neutralizing status to one or a plurality of subjects known to be infected with the SARS-CoV-2 virus; (c) assessing the SARS-CoV-2 virus infection in the cell or subject following the contacting and administering, respectively; and (d) determining from the assessment a presence and/or level of efficacy of the SARS-CoV-2 anti-virus antibody treatment of the SARS-CoV-2 infection in the cell or subject, respectively, wherein one or both of the presence and efficacy identifies the SARS-CoV-2 anti-virus antibody as a candidate antibody to treat an infection by the SARS-CoV-2 virus.

According to another aspect of the invention, an enzyme-linked lectin assay (ELLA) is provided, the ELLA including a plurality of preselected lectins that bind a virus protein. In some embodiments, the virus protein is a spike protein. In some embodiments, the virus is an RNA virus. In certain embodiments, the virus is an enveloped virus. In certain embodiments, the virus is a positive single-stranded RNA virus, and optionally is a member of the viral family Astroviridae, Caliciviridae Picornaviridae, Coronaviridae, Flaviviridae, Retroviridae, or Togaviridae. In some embodiments, the virus is a MERS-CoV virus or a SARS-CoV virus. In some embodiments, the virus is a SARS-CoV-1, SARS-CoV-2 virus, or an infective functional variant thereof. In certain embodiments, the virus is a negative single-stranded RNA virus, and optionally is a member of the viral family Arenaviridae, Bunyaviridae, Filoviridae, Orthomyxoviridae, Paramyxoviridae, or Rhabdoviridae. In certain embodiments, the plurality of preselected lectins includes two or more of: Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1 molecules. In certain embodiments, the plurality of preselected lectins includes 2, 3, 4, 5, 6, 7, 8, 9, 10, 11, 12, 13, 14, 15, 16, 17, 18, 19, 20, 21, 22, 23, 24, 25, 26, 27, 28, or 29 of: Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1 molecules. In some embodiments the plurality of preselected lectins includes each of: Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1 molecules. In some embodiments, the virus is a SARS-CoV-2 virus and the preselected lectin molecules contacting the virion protein include MALI, RPL-αMan, RPL-Man2, PNA, RPL-Gal1, Jacalin, RPL-αGal, RPL-Sia1, RPL-Fuc1, DBA, and LTL molecules. In certain embodiments, the plurality of the preselected lectin molecules includes MALI, RPL-αMan, RPL-Man2, PNA, RPL-Gal1, Jacalin, RPL-αGal, RPL-Sia1, RPL-Fuc1, DBA, and LTL molecules. In some embodiments, the virus is a SARS-CoV-2 virus and the plurality of preselected lectin molecules comprise: ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1 molecules.

According to another aspect of the invention, a method of identifying a virus-neutralization status of a candidate antiviral agent is provided, the method including: (a) contacting a virion of a virus that has previously contracted an anti-virus antibody, with one or more preselected glycan-binding agents, wherein the contacting results in a virion/glycan-binding agent complex; (b) contacting the virion/glycan-binding agent complex with a candidate antiviral agent that is a candidate to block infection with the virus; and (c) determining one or more binding characteristics of the preselected glycan-binding agent(s) with the antibody-contacted virion subsequent to the contacting with the candidate antiviral agent, wherein the determined one or more binding characteristics identifies the virus-neutralization status of the antiviral agent. In some embodiments, the glycan-binding agent includes a protein. In certain embodiments, the glycan-binding agent is one or more of a naturally occurring glycan-binding agent, a variant of a glycan-binding agent, a functional fragment of a glycan-binding agent, a recombinant glycan-binding agent, a functional fragment of a recombinant glycan-binding agent, an engineered glycan-binding agent, and an engineered fragment of a glycan-binding agent. In certain embodiments, the glycan-binding agent is a glycan-binding protein, optionally is a glycan-binding antibody, a glycan-binding antibody fragment, a lectin, or a glycan-binding nanobody that binds a specific glycan epitope.

### BRIEF DESCRIPTION OF THE DRAWINGS

FIG. 1A-E provides schematic diagrams and a bar chart illustrating glycosylation sites mapped onto the SARS-CoV-2 Spike glycoprotein. FIG. 1A is a diagram showing glycan processing following protein translation leads to sequence microheterogeneity at N- and O-glycosites (N-X-S/T and S/T, respectively) where X is any amino acid except proline. FIG. 1B is a diagram showing the multidomain WT and Delta Spike glycoproteins contain the receptor binding domain (RBD). Predictions suggest 22 N-glycans (gray, diamonds indicate RBD) and two 0-glycans (stars) are present in each monomer of the trimeric Spike glycoprotein. FIG. 1C shows sequence alignment of WT and Delta Spike RBD region showing S323/T325 (top alignment), N331/343 (top alignment), and Delta mutations L452R and T478K (lower alignment). Top alignment shows WT sequence (SEQ ID NO: 6) and Delta sequence (SEQ ID NO: 6). Lower alignment shows WT sequence (SEQ ID NO: 7) and Delta sequence (SEQ ID NO: 8). FIG. 1D provides an interaction map of 46 distinct glycans present on FL-Spike and 24 plant and fungal lectins. Predicted strong (triangles), intermediate (diamonds), and weak (stars) binders have decreasing number of shared glycans. FIG. 1E shows lectin fingerprints for RBD and FL-Spike.

FIG. 2 is a lectin correlation map of overlapping glycans from the CFG mammalian glycan array of 611 glycans plotted using the GLAD tool. FL-Spike glycan Pearson correlation analysis with lectin specificity used to predict lectin binders. Correlation ≥0.1 considered high, between 0.1 and 0 considered intermediate and negative values considered weak.

FIG. 3 provides schematic diagram illustrating a lectin fingerprinting assay. FL-Spike or RBD run against each 24 plant or 6 prokaryotic biotinylated lectins in ELLA assay to generate unique protein fingerprints.

FIG. 4 is a bar graph illustrating dose-dependent binding of 24 plant and fungal lectins against immobilized FL-Spike (10 μg/mL). Each lectin was added at 10, 1 and 0.1 mg/mL in duplicate and showed dose-depending binding. Fingerprints are representative of data from three individual experiments.

FIG. 5A-C provides graphs and a photographic image of results of studies of PNGase F treatment of (FIG. 5A) RBD and (FIG. 5B) FL-Spike show significant reduction in lectin binding. FIG. 5C is a stain-free SDS-PAGE of PNGase F treated RBD and FL-Spike does not completely remove N-glycans. Fold decrease bar graphs are representative of two individual experiments.

FIG. 6 is a bar graph showing lectin fingerprints from recombinant FL-Spike and RBD from HEK and Insect cells have distinct signatures. ELLA assay with immobilized FL-Spike, HEK-expressed RBD, or Insect-expressed RBD were treated with a panel of 10 lectins to determine differences in glycan heterogeneity. Lectin fingerprints are representative of two individual experiments. In FIG. 6, in the set of three bars above each lectin on the horizontal axis, the left bar is FL-Spike, the middle bar is HEK RBD, and the right bar is Insect RBD.

FIG. 7A-E provides schematic diagrams, a graph and a dendrogram illustrating results indicating that lectin fingerprints reveal neutralizing potential of pre-vaccinated patient antibodies and patient-derived sera. FIG. 7A is a schematic diagram showing host infection by SARS-CoV-2 elicits a rapid humoral response producing either neutralizing or non-neutralizing antibodies. FIG. 7B is a schematic diagram illustrating a competitive ELLA assay to reveal unique lectin fingerprints. FIG. 7C is a graph showing log (fold change) upon treatment of immobilized RBD and FL-Spike containing 24 plant and fungal lectins with MM43 commercial anti-RBD antibody (1 μg/mL) in parallel. In FIG. 7C, in the set of two bars above each lectin on the horizontal axis, the left bar is RBD, and the right bar is FL-Spike. FIG. 7D is a patient sera metadata table used in blinded study (n=8) indicate seropositivity and estimated RBD abundance. Serum dilution in pseudovirus inhibition assay performed with pseudotyped virus indicates competitive ID50 values. FIG. 7 E is a dendrogram displaying hierarchical clustering analysis of immobilized RBD with bound plant, fungal, and prokaryotic lectins (1 μg/mL) treated with convalescent patient sera (1:100 dilution) Distinct and separate lectin fingerprints were observed for each sample class. Dendrograms from neutralizing serum (n=2 row 1, 2) and non-neutralizing (rows 3-6, n=4). Hierarchical analysis represents data from two separate experiments.

FIG. 8A-B are bar graphs showing results of study in which immobilized neutralizing (MM43, B38, 1 μg/mL) and non-neutralizing (CR3022, 1 μg/mL) antibodies were evaluated for binding against a panel of plant, prokaryotic, and fungal lectins (1 μg/mL) using an ELISA. Antibodies were tested alongside Spike RBD (10 μg/mL) and a BSA only control (black). Dotted lines represent background binding signal threshold based on BSA controls. Data are arranged by high (FIG. 8A) and moderate to low (FIG. 8B) lectin binding to RBD. Results are representative of duplicate experiments. In FIGS. 8A and B in the set of five bars above each lectin on the horizontal axis, the order of bars in each set from left to right is: BSA, 3022, B38, MM43, and RBD.

FIG. 9A-C provides graphs and a dendrogram illustrating that competitive lectin fingerprints indicate unique signatures for neutralizing antibodies. FIG. 9A is a graph of results of a competitive ELLA assay showing Log (fold change) upon treatment of immobilized RBD and FIG. 9B is a graph showing FL-Spike containing 28 plant, fungal, and prokaryotic lectins with MM43 commercial anti-RBD antibody (1 μg/mL) in parallel. FIG. 9C is a dendrogram displaying hierarchical clustering analysis of immobilized RBD with bound plant and prokaryotic lectins (1 μg/mL) treated with convalescent patient and commercial antibodies (n=7, 1 μg/mL) Dendrograms from highly potent neutralizing antibody (labeled as (i), n=1), weakly neutralizing, non-orthosteric neutralizing, and non-neutralizing monoclonal antibodies (mAbs) (labelled as (ii) n=3), commercial neutralizing mAb (Labeled as (iii), n=1) and cross reactive anti-S1 (labelled as (iv), n=2) antibody with SARS-CoV-1 indicate distinct and separate lectin fingerprints were observed for each sample. Heatmap plotted as a fold change of lectin signal and is representative of two experiments.

FIG. 10A-C provides a schematic diagram and graphs illustrating competitive lectin fingerprint assay using (FIG. 10A) neutralizing antibodies against immobilized RBD. Dose-dependent binding of recombinant prokaryotic lectins (RPL) at (10, 1 and 0.1 μg/mL) to (FIG. 10B) FL-Spike and (FIG. 10C) RBD. Prokaryotic lectin fingerprints shown are representative of two individual experiments. In FIG. 10B-C in the set of three bars above each RPL lectin on the horizontal axis, the left bar is 10 μg/mL, the middle bar is 1 μg/mL, and the right bar is 0.1 μg/mL.

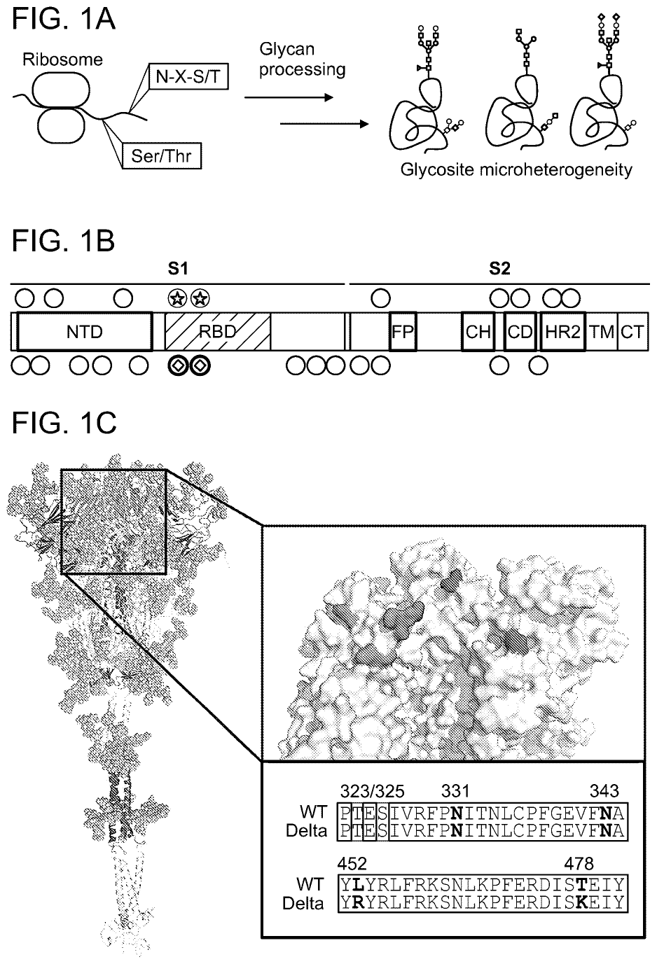

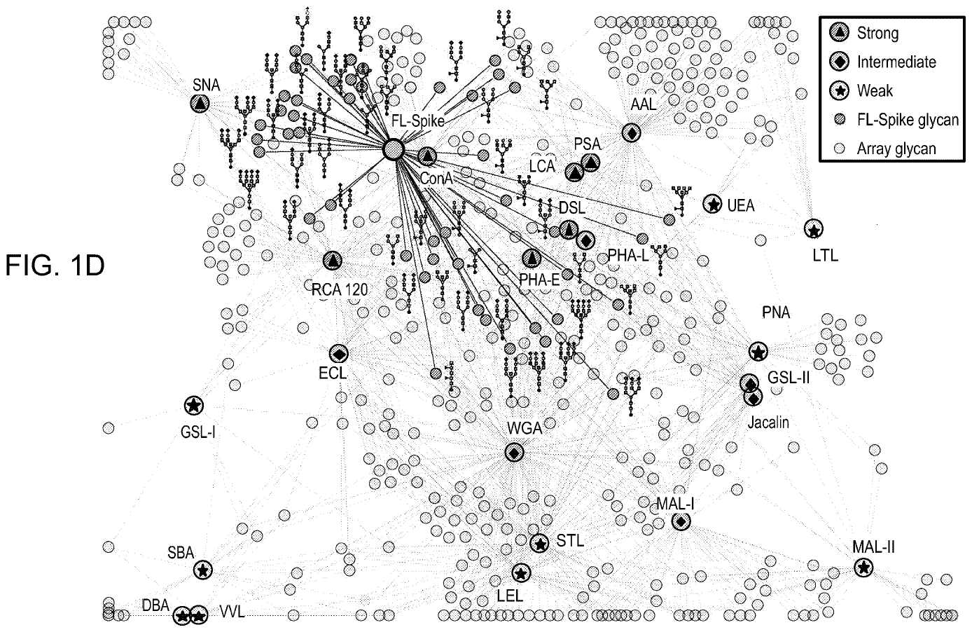

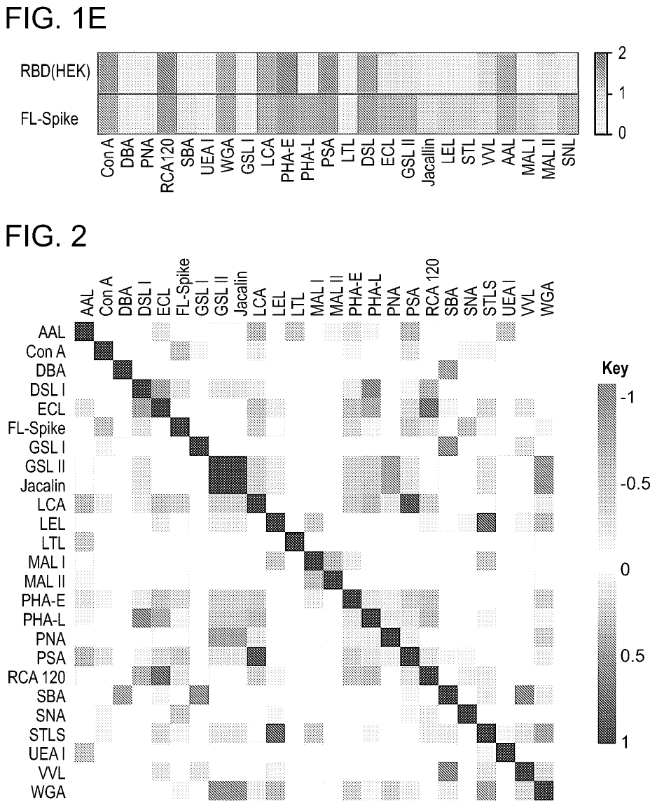

FIG. 1 is a graph of principal component analysis of lectin displacement by neutralizing (triangle) and non-neutralizing (star) convalescent patient sera (1:100 dilution) on immobilized RBD. Distinct and separate lectin fingerprints were observed for each sample class. Analysis represents data from two separate experiments.

FIG. 12A-I provides tables, graphs and dendrograms illustrating relation of O-glycosylation differences of VOCs to antibody recognition in vaccinated patient sera. FIG. 12A is a table showing human patient sera (n=16) and their neutralization potential (ID₅₀) against WT and Delta VOC in a pseudovirus neutralization assay. FIG. 12B is a graph showing results of patient sera binding to Spike RBD variants assessed by ELISA. Darker bars are Delta, lighter bars are wild type. FIG. 12C are graphs showing correlation of neutralization data to normalized ELISA data of WT (left) and Delta (right). FIG. 12D is a schematic diagram illustrating O-Glycosite and trypsin digestion fragment analyzed by glycoproteomics. Darker circles are Delta, lighter circles are wild type. FIG. 12E is a graph showing results of differential scanning fluorimetry of WT and Delta Spike RBD. FIG. 12F is a graph of a lectin fingerprinting comparison of WT over Delta glycans with no PNGase F and FIG. 12G PNGase F treatment. FIG. 12H are pie charts of glycoproteomics data from WT (left) and Delta (right) Spike RBD showing the three most abundant glycans detected. FIG. 12 I are dendrograms displaying hierarchical clustering analysis of immobilized WT (left) or Delta (right) Spike RBD with bound plant, fungal, and prokaryotic lectins (1 μg/mL) and vaccinated patient sera (BNT162b2, 1:100 dilution). Dendrograms from neutralizing serum (n=4) and non-neutralizing (n=2), and intermediate (n=1). Hierarchical analysis represents data from two separate experiments.

FIG. 13A-B provide a dendrogram and graph illustrating results indicating that a mRNA vaccine strategy yields distinct lectin fingerprints. FIG. 13A is a dendrogram displaying hierarchical clustering analysis of immobilized Delta Spike RBD with bound plant, fungal, and prokaryotic lectins (1 μg/mL) treated with vaccinated patient sera BNT162b2 (n=7) or mRNA-1273 (n=9) diluted 1:100. Hierarchical analysis represents data from two separate experiments. FIG. 13B is a graph showing results of correlation analysis of Serum 15 and Serum 14 (mRNA-1273) with enriched lectins indicated with darker circles, and darker circles with arrows, respectively (left). Glycan specificity of enriched lectins (right).

### BRIEF DESCRIPTION OF SEQUENCES

SEQ ID NO: 1 is amino acid sequence of RBD treated with HRV3c to remove a polyHis and SBP tag. Italicized amino acids show the remnant of an HRV 3C cleavage site.

MRVQPTESIVRFPNITNLCPFGEVENATRFASVYAWNRKRISNCVADYS
VLYNSASFSTFKCYGVSPTKLNDLCFTNVYADSFVIRGDEVRQIAPGQT
GKIADYNYKLPDDFTGCVIAWNSNNLDSKVGGNYNYLYRLFRKSNLKPF
ERDISTEIYQAGSTPCNGVEGENCYFPLQSYGFQPTNGVGYQPYRVVVL
SFELLHAPATVCGPKKGAGSSLEVLFQ.

SEQ ID NO: 2 is the amino acid sequence of viral spike protein for SARS-CoV-2 (Genbank accession number is MN975262.1).

MESLVPGFNEKTHVQLSLPVLQVRDVLVRGFGDSVEEVLSEARQHLKDGTCGLVEVEK
GVLPQLEQPYVFIKRSDARTAPHGHVMVELVAELEGIQYGRSGETLGVLVPHVGEIPVA
YRKVLLRKNGNKGAGGHSYGADLKSFDLGDELGTDPYEDFQENWNTKHSSGVTRELM
RELNGGAYTRYVDNNFCGPDGYPLECIKDLLARAGKASCTLSEQLDFIDTKRGVYCCRE
HEHEIAWYTERSEKSYELQTPFEIKLAKKFDTFNGECPNFVFPLNSIIKTIQPRVEKKKLD
GFMGRIRSVYPVASPNECNQMCLSTLMKCDHCGETSWQTGDFVKATCEFCGTENLTKE
GATTCGYLPQNAVVKIYCPACHNSEVGPEHSLAEYHNESGLKTILRKGGRTIAFGGCVF
SYVGCHNKCAYWVPRASANIGCNHTGVVGEGSEGLNDNLLEILQKEKVNINIVGDFKL
NEEIAIILASFSASTSAFVETVKGLDYKAFKQIVESCGNFKVTKGKAKKGAWNIGEQKSIL
SPLYAFASEAARVVRSIFSRTLETAQNSVRVLQKAAITILDGISQYSLRLIDAMMFTSDLA
TNNLVVMAYITGGVVQLTSQWLTNIFGTVYEKLKPVLDWLEEKFKEGVEFLRDGWEIV
KFISTCACEIVGGQIVTCAKEIKESVQTFFKLVNKFLALCADSIIIGGAKLKALNLGETFVT
HSKGLYRKCVKSREETGLLMPLKAPKEIIFLEGETLPTEVLTEEVVLKTGDLQPLEQPTSE
AVEAPLVGTPVCINGLMLLEIKDTEKYCALAPNMMVTNNTFTLKGGAPTKVTFGDDTVI
EVQGYKSVNITFELDERIDKVLNEKCSAYTVELGTEVNEFACVVADAVIKTLQPVSELLT
PLGIDLDEWSMATYYLFDESGEFKLASHMYCSFYPPDEDEEEGDCEEEEFEPSTQYEYG
TEDDYQGKPLEFGATSAALQPEEEQEEDWLDDDSQQTVGQQDGSEDNQTTTIQTIVEVQ
PQLEMELTPVVQTIEVNSFSGYLKLTDNVYIKNADIVEEAKKVKPTVVVNAANVYLKH
GGGVAGALNKATNNAMQVESDDYIATNGPLKVGGSCVLSGHNLAKHCLHVVGPNVN
KGEDIQLLKSAYENFNQHEVLLAPLLSAGIFGADPIHSLRVCVDTVRTNVYLAVEDKNL
YDKLVSSFLEMKSEKQVEQKIAEIPKEEVKPFITESKPSVEQRKQDDKKIKACVEEVTTTL
EETKFLTENLLLYIDINGNLHPDSATLVSDIDITFLKKDAPYIVGDVVQEGVLTAVVIPTK
KAGGTTEMLAKALRKVPTDNYITTYPGQGLNGYTVEEAKTVLKKCKSAFYILPSIISNEK
QEILGTVSWNLREMLAHAEETRKLMPVCVETKAIVSTIQRKYKGIKIQEGVVDYGARFY
FYTSKTTVASLINTLNDLNETLVTMPLGYVTHGLNLEEAARYMRSLKVPATVSVSSPDA
VTAYNGYLTSSSKTPEEHFIETISLAGSYKDWSYSGQSTQLGIEFLKRGDKSVYYTSNPTT
FHLDGEVITFDNLKTLLSLREVRTIKVFTTVDNINLHTQVVDMSMTYGQQFGPTYLDGA
DVTKIKPHNSHEGKTFYVLPNDDTLRVEAFEYYHTTDPSFLGRYMSALNHTKKWKYPQ
VNGLTSIKWADNNCYLATALLTLQQIELKFNPPALQDAYYRARAGEAANFCALILAYCN
KTVGELGDVRETMSYLFQHANLDSCKRVLNVVCKTCGQQQTTLKGVEAVMYMGTLS
YEQFKKGVQIPCTCGKQATKYLVQQESPFVMMSAPPAQYELKHGTFTCASEYTGNYQC
GHYKHITSKETLYCIDGALLTKSSEYKGPITDVFYKENSYTTTIKPVTYKLDGVVCTEIDP
KLDNYYKKDNSYFTEQPIDLVPNQPYPNASFDNFKFVCDNIKFADDLNQLTGYKKPASR
ELKVTFFPDLNGDVVAIDYKHYTPSFKKGAKLLHKPIVWHVNNATNKATYKPNTWCIR
CLWSTKPVETSNSFDVLKSEDAQGMDNLACEDLKPVSEEVVENPTIQKDVLECNVKTTE
VVGDIILKPANNSLKITEEVGHTDLMAAYVDNSSLTIKKPNELSRVLGLKTLATHGLAAV
NSVPWDTIANYAKPFLNKVVSTTTNIVTRCLNRVCTNYMPYFFTLLLQLCTFTRSTNSRI
KASMPTTIAKNTVKSVGKFCLEASFNYLKSPNFSKLINIIIWFLLLSVCLGSLIYSTAALGV
LMSNLGMPSYCTGYREGYLNSTNVTIATYCTGSIPCSVCLSGLDSLDTYPSLETIQITISSF
KWDLTAFGLVAEWFLAYILFTRFFYVLGLAAIMQLFFSYFAVHFISNSWLMWLIINLVQ
MAPISAMVRMYIFFASFYYVWKSYVHVVDGCNSSTCMMCYKRNRATRVECTTIVNGV
RRSFYVYANGGKGFCKLHNWNCVNCDTFCAGSTFISDEVARDLSLQFKRPINPTDQSSY
IVDSVTVKNGSIHLYFDKAGQKTYERHSLSHFVNLDNLRANNTKGSLPINVIVFDGKSKC
EESSAKSASVYYSQLMCQPILLLDQALVSDVGDSAEVAVKMFDAYVNTFSSTFNVPME
KLKTLVATAEAELAKNVSLDNVLSTFISAARQGFVDSDVETKDVVECLKLSHQSDIEVT
GDSCNNYMLTYNKVENMTPRDLGACIDCSARHINAQVAKSHNIALIWNVKDFMSLSEQ
LRKQIRSAAKKNNLPFKLTCATTRQVVNVVTTKIALKGGKIVNNWLKQLIKVTLVFLFV
AAIFYLITPVHVMSKHTDFSSEIIGYKAIDGGVTRDIASTDTCFANKHADFDTWFSQRGG
SYTNDKACPLIAAVITREVGFVVPGLPGTILRTTNGDFLHFLPRVFSAVGNICYTPSKLIE
YTDFATSACVLAAECTIFKDASGKPVPYCYDTNVLEGSVAYESLRPDTRYVLMDGSIIQF
PNTYLEGSVRVVTTFDSEYCRHGTCERSEAGVCVSTSGRWVLNNDYYRSLPGVFCGVD
AVNLLTNMFTPLIQPIGALDISASIVAGGIVAIVVTCLAYYFMRFRRAFGEYSHVVAFNTL
LFLMSFTVLCLTPVYLFLPGVYSVIYLYLTFYLTNDVSFLAHIQWMVMFTPLVPFWITIA
YIICISTKHFYWFFSNYLKRRVVFNGVSFSTFEEAALCTFLLNKEMYLKLRSDVLLPLTQ
YNRYLALYNKYKYFSGAMDTTSYREAACCHLAKALNDFSNSGSDVLYQPPQTSITSAV
LQSGFRKMAFPSGKVEGCMVQVTCGTTTLNGLWLDDVVYCPRHVICTSEDMLNPNYE
DLLIRKSNHNFLVQAGNVQLRVIGHSMQNCVLKLKVDTANPKTPKYKFVRIQPGQTFSV
LACYNGSPSGVYQCAMRPNFTIKGSFLNGSCGSVGFNIDYDCVSFCYMHHMELPTGVH
AGTDLEGNFYGPFVDRQTAQAAGTDTTITVNVLAWLYAAVINGDRWFLNRFTTTLNDF
NLVAMKYNYEPLTQDHVDILGPLSAQTGIAVLDMCASLKELLQNGMNGRTILGSALLE
DEFTPFDVVRQCSGVTFQSAVKRTIKGTHHWLLLTILTSLLVLVQSTQWSLFFFLYENAF
LPFAMGIIAMSAFAMMFVKHKHAFLCLFLLPSLATVAYFNMVYMPASWVMRIMTWLD
MVDTSLSGFKLKDCVMYASAVVLLILMTARTVYDDGARRVWTLMNVLTLVYKVYYG
NALDQAISMWALIISVTSNYSGVVTTVMFLARGIVEMCVEYCPIFFITGNTLQCIMLVYC
FLGYFCTCYFGLFCLLNRYFRLTLGVYDYLVSTQEFRYMNSQGLLPPKNSIDAFKLNIKL
LGVGGKPCIKVATVQSKMSDVKCTSVVLLSVLQQLRVESSSKLWAQCVQLHNDILLAK
DTTEAFEKMVSLLSVLLSMQGAVDINKLCEEMLDNRATLQAIASEFSSLPSYAAFATAQ
EAYEQAVANGDSEVVLKKLKKSLNVAKSEFDRDAAMQRKLEKMADQAMTQMYKQA
RSEDKRAKVTSAMQTMLFTMLRKLDNDALNNIINNARDGCVPLNIIPLTTAAKLMVVIP
DYNTYKNTCDGTTFTYASALWEIQQVVDADSKIVQLSEISMDNSPNLAWPLIVTALRAN
SAVKLQNNELSPVALRQMSCAAGTTQTACTDDNALAYYNTTKGGRFVLALLSDLQDL
KWARFPKSDGTGTIYTELEPPCRFVTDTPKGPKVKYLYFIKGLNNLNRGMVLGSLAATV
RLQAGNATEVPANSTVLSFCAFAVDAAKAYKDYLASGGQPITNCVKMLCTHTGTGQAI
TVTPEANMDQESFGGASCCLYCRCHIDHPNPKGFCDLKGKYVQIPTTCANDPVGFTLKN
TVCTVCGMWKGYGCSCDQLREPMLQSADAQSFLNRVCGVSAARLTPCGTGTSTDVVY
RAFDIYNDKVAGFAKFLKTNCCRFQEKDEDDNLIDSYFVVKRHTFSNYQHEETIYNLLK
DCPAVAKHDFFKFRIDGDMVPHISRQRLTKYTMADLVYALRHFDEGNCDTLKEILVTY
NCCDDDYFNKKDWYDFVENPDILRVYANLGERVRQALLKTVQFCDAMRNAGIVGVLT
LDNQDLNGNWYDFGDFIQTTPGSGVPVVDSYYSLLMPILTLTRALTAESHVDTDLTKPY
IKWDLLKYDFTEERLKLFDRYFKYWDQTYHPNCVNCLDDRCILHCANFNVLFSTVFPPT
SFGPLVRKIFVDGVPFVVSTGYHFRELGVVHNQDVNLHSSRLSFKELLVYAADPAMHA
ASGNLLLDKRTTCFSVAALTNNVAFQTVKPGNFNKDFYDFAVSKGFFKEGSSVELKHFF
FAQDGNAAISDYDYYRYNLPTMCDIRQLLFVVEVVDKYFDCYDGGCINANQVIVNNLD
KSAGFPFNKWGKARLYYDSMSYEDQDALFAYTKRNVIPTITQMNLKYAISAKNRARTV
AGVSICSTMTNRQFHQKLLKSIAATRGATVVIGTSKFYGGWHNMLKTVYSDVENPHLM
GWDYPKCDRAMPNMLRIMASLVLARKHTTCCSLSHRFYRLANECAQVLSEMVMCGGS
LYVKPGGTSSGDATTAYANSVENICQAVTANVNALLSTDGNKIADKYVRNLQHRLYEC
LYRNRDVDTDFVNEFYAYLRKHFSMMILSDDAVVCFNSTYASQGLVASIKNFKSVLYY
QNNVFMSEAKCWTETDLTKGPHEFCSQHTMLVKQGDDYVYLPYPDPSRILGAGCFVDD
IVKTDGTLMIERFVSLAIDAYPLTKHPNQEYADVFHLYLQYIRKLHDELTGHMLDMYSV
MLTNDNTSRYWEPEFYEAMYTPHTVLQAVGACVLCNSQTSLRCGACIRRPFLCCKCCY
DHVISTSHKLVLSVNPYVCNAPGCDVTDVTQLYLGGMSYYCKSHKPPISFPLCANGQVF
GLYKNTCVGSDNVTDFNAIATCDWTNAGDYILANTCTERLKLFAAETLKATEETFKLSY
GIATVREVLSDRELHLSWEVGKPRPPLNRNYVFTGYRVTKNSKVQIGEYTFEKGDYGDA
VVYRGTTTYKLNVGDYFVLTSHTVMPLSAPTLVPQEHYVRITGLYPTLNISDEFSSNVA
NYQKVGMQKYSTLQGPPGTGKSHFAIGLALYYPSARIVYTACSHAAVDALCEKALKYL
PIDKCSRIIPARARVECFDKFKVNSTLEQYVFCTVNALPETTADIVVFDEISMATNYDLSV
VNARLRAKHYVYIGDPAQLPAPRTLLTKGTLEPEYFNSVCRLMKTIGPDMFLGTCRRCP
AEIVDTVSALVYDNKLKAHKDKSAQCFKMFYKGVITHDVSSAINRPQIGVVREFLTRNP
AWRKAVFISPYNSQNAVASKILGLPTQTVDSSQGSEYDYVIFTQTTETAHSCNVNRFNV
AITRAKVGILCIMSDRDLYDKLQFTSLEIPRRNVATLQAENVTGLFKDCSKVITGLHPTQ
APTHLSVDTKFKTEGLCVDIPGIPKDMTYRRLISMMGFKMNYQVNGYPNMFITREEAIR
HVRAWIGFDVEGCHATREAVGTNLPLQLGFSTGVNLVAVPTGYVDTPNNTDFSRVSAK
PPPGDQFKHLIPLMYKGLPWNVVRIKIVQMLSDTLKNLSDRVVFVLWAHGFELTSMKY
FVKIGPERTCCLCDRRATCFSTASDTYACWHHSIGFDYVYNPFMIDVQQWGFTGNLQSN
HDLYCQVHGNAHVASCDAIMTRCLAVHECFVKRVDWTIEYPIIGDELKINAACRKVQH
MVVKAALLADKFPVLHDIGNPKAIKCVPQADVEWKFYDAQPCSDKAYKIEELFYSYAT
HSDKFTDGVCLFWNCNVDRYPANSIVCRFDTRVLSNLNLPGCDGGSLYVNKHAFHTPA
FDKSAFVNLKQLPFFYYSDSPCESHGKQVVSDIDYVPLKSATCITRQNLGGAVCRHHAN
EYRLYLDAYNMMISAGFSLWVYKQFDTYNLWNTFTRLQSLENVAFNVVNKGHFDGQQ
GEVPVSIINNTVYTKVDGVDVELFENKTTLPVNVAFELWAKRNIKPVPEVKILNNLGVDI
AANTVIWDYKRDAPAHISTIGVCSMTDIAKKPTETICAPLTVFFDGRVDGQVDLFRNAR
NGVLITEGSVKGLQPSVGPKQASLNGVTLIGEAVKTQFNYYKKVDGVVQQLPETYFTQS
RNLQEFKPRSQMEIDFLELAMDEFIERYKLEGYAFEHIVYGDFSHSQLGGLHLLIGLAKR
FKESPFELEDFIPMDSTVKNYFITDAQTGSSKCVCSVIDLLLDDFVEIIKSQDLSVVSKVV
KVTIDYTEISFMLWCKDGHVETFYPKLQSSQAWQPGVAMPNLYKMQRMLLEKCDLQN
YGDSATLPKGIMMNVAKYTQLCQYLNTLTLAVPYNMRVIHFGAGSDKGVAPGTAVLR
QWLPTGTLLVDSDLNDFVSDADSTLIGDCATVHTANKWDLIISDMYDPKTKNVTKEND
SKEGFFTYICGFIQQKLALGGSVAIKITEHSWNADLYKLMGHFAWWTAFVTNVNASSSE
AFLIGCNYLGKPREQIDGYVMHANYIFWRNTNPIQLSSYSLFDMSKFPLKLRGTAVMSL
KEGQINDMILSLLSKGRLIIRENNRVVISSDVLVNN.

SEQ ID NO: 3 is SEQ ID NO: 2 in which the furin cleavage site obliterated and two proline mutations are included to stabilize it in a prefusion conformation. The first 15 amino acids are the signal peptide (underlined); the amino acids GSAS (underlined) make up the furin cleavage site; and the PP amino acids (underlined) are two proline mutations.

MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQT
LLALHRSYLTPGDSSSGWTAGAAAYYVGYLQPRTFLLKYNENGTITDAVDCALDPLSET
KCTLKSFTVEKGIYQTSNFRVQPTESIVRFPNITNLCPFGEVFNATRFASVYAWNRKRISN
CVADYSVLYNSASFSTFKCYGVSPTKLNDLCFTNVYADSFVIRGDEVRQIAPGQTGKIA
DYNYKLPDDFTGCVIAWNSNNLDSKVGGNYNYLYRLFRKSNLKPFERDISTEIYQAGST
PCNGVEGFNCYFPLQSYGFQPTNGVGYQPYRVVVLSFELLHAPATVCGPKKSTNLVKN
KCVNFNFNGLTGTGVLTESNKKFLPFQQFGRDIADTTDAVRDPQTLEILDITPCSFGGVS
VITPGTNTSNQVAVLYQDVNCTEVPVAIHADQLTPTWRVYSTGSNVFQTRAGCLIGAEH
VNNSYECDIPIGAGICASYQTQTNSPGSASSVASQSIIAYTMSLGAENSVAYSNNSIAIPTN
FTISVTTEILPVSMTKTSVDCTMYICGDSTECSNLLLQYGSFCTQLNRALTGIAVEQDKNT
QEVFAQVKQIYKTPPIKDFGGFNFSQILPDPSKPSKRSFIEDLLFNKVTLADAGFIKQYGD
CLGDIAARDLICAQKFNGLTVLPPLLTDEMIAQYTSALLAGTITSGWTFGAGAALQIPFA
MQMAYRFNGIGVTQNVLYENQKLIANQFNSAIGKIQDSLSSTASALGKLQDVVNQNAQ
ALNTLVKQLSSNFGAISSVLNDILSRLDPPEAEVQIDRLITGRLQSLQTYVTQQLIRAAEIR
ASANLAATKMSECVLGQSKRVDFCGKGYHLMSFPQSAPHGVVFLHVTYVPAQEKNFTT
APAICHDGKAHFPREGVFVSNGTHWFVTQRNFYEPQIITTDNTFVSGNCDVVIGIVNNTV
YDPLQPELDSFKEELDKYFKNHTSPDVDLGDISGINASVVNIQKEIDRLNEVAKNLNESLI
DLQELGKYEQGSGYIPEAPRDGQAYVRKDGEWVLLSTFLGRSLEVLFQ.

SEQ ID NO: 4 is the signal peptide of SEQ ID NO: 3

MFVFLVLLPLVSSQC.

SEQ ID NO: 5 is the furin cleavage site sequence in SEQ ID NO: 3.

GSAS.

SEQ ID NO: 6 is PTESIVRFPNITNLCPFGEVFNA.

SEQ ID NO: 7 is: YLYRLFRKSNLKPFERDISTEIY.

SEQ ID NO: 8 is YRYRLFRKSNLKPFERDISKEIY.

### DETAILED DESCRIPTION

Methods of the invention, in part, relate to novel tools to quickly and easily report on changes in Spike glycosylation among viral variants and provide insights into how viruses such as SARS-CoV-2 modify their glycans to evade immune surveillance. Moreover, methods of the invention permit these glycan reporters to be leveraged to generate recognition patterns, or fingerprints, from neutralizing and non-neutralizing antibodies. Using embodiments of methods of the invention, unique fingerprints generated by antibodies with distinct mechanisms of action (e.g., direct competition with receptor and co-receptor binding sites) can be identified and used as standards for evaluating whether patient sera contain antibodies with similar properties. Methods set forth herein describe a lectin fingerprinting strategy that can be used to predict the neutralization potential of antibodies against WT and Delta SARS-CoV-2 variants. Embodiments of methods of the invention can be used to determine how viruses utilize glycosylation to subvert host immune defenses.

Viruses co-opt host cellular glycosylation pathways to decorate their surface proteins. Viral glycosylation can influence host interactions and subvert immune recognition. It has now been discovered that carbohydrate-binding proteins (lectins) compete with antibodies for binding to viral glycosites, and thereby can be used to determine viral neutralization. Described herein are lectin fingerprinting methods that can be used to rapidly profile protein glycosylation state and its impact on antibody recognition of the viral glycoprotein. Non-limiting examples of lectin fingerprinting methods described herein includes the highly glycosylated SARS-CoV-2 Spike protein as a model system. Curated sets of prokaryotic, fungal, and plant lectins may be used in the system to detect distinct glycosylation fingerprints of recombinant Spike protein and its receptor-binding domain (RBD). It has been determined that in the presence of antibodies or convalescent patient sera, these lectin fingerprints are perturbed. Methods of the invention, in part, include lectin fingerprints that can distinguish virus-neutralizing versus non-virus-neutralizing antibodies. Thus, the invention, in some aspects includes using lectin fingerprinting to report on the neutralization potential of antibodies that bind a critical viral glycoprotein. The term “virus neutralization” will be understood to mean inhibiting activity of the virus, which may include, but is not limited to one or more of inhibiting binding of the virion to a cell, inhibiting entry of a viral particle into a cell, inhibiting viral reverse transcription, and inhibiting viral integration into a cell, inhibiting release of virion particles from a cell, etc.

The Spike glycoprotein is a multidomain, class I fusion protein that is a homotrimer present on the virion surface; it is densely glycosylated, containing two putative O-glycosylation and 22 predicted N-glycosylation sites per monomer (FIG. 1B) [Shajahan, A., et al., Glycobiology (2020) doi:10.1093/glycob/cwaa042; Watanabe, Y., et al., Science eabb9983 (2020) doi:10.1126/science.abb9983]. Acute and specific antibody production occurs rapidly in response to the highly immunogenic Spike during SARS-CoV-2 infection [Long, Q.-X. et al. at. Med. 26, 845-848 (2020)]. Many neutralizing and non-neutralizing antibodies bind the RBD at the ACE2 binding site [Brouwer, P. J. M. et al. Science 369, 643-650 (2020); Casalino, L. et al. ACS Cent. Sci. (2020) doi:10.1021/acscentsci.0c01056]. Proximal to this binding site are the glycosylated residues N331 and N343, which contact neutralizing antibodies from patients convalescent for other coronaviruses, including MERS-CoV and SARS-CoV-117. Neutralizing antibodies that bind the Spike protein can also engage regions other than the RBD and neutralize through cryptic mechanisms. The Spike protein is glycosylated and is the major glycoprotein present on the virion surface, and it has now been determined that the accessibility of different glycans can be used to determine and report on antibody recognition of the spike protein. The invention, in part, includes methods of determining variations in accessibility of specific lectins to these glycans, and assessing the determined variations to identify the capability of an antibody that has contacted the virus, to neutralize the virus.

### Virus, Infections, and Symptoms

A viral infection, which may also be referred to as a viral disease or infection by a virus, may occur in a subject when a pathogenic virus is present in the subject and infectious virus particles (virions) attach to and enter one or more of the subject's cells. A subject infected with a virus may also be referred to herein as a “host” subject. Presence of a viral infection in a subject may result in the subject's immune system generating anti-virus antibodies that recognize and bind one or more proteins in the virus, also referred to herein as virus proteins. Numerous different viruses are known to infect subjects and cells. Categories of infective viruses include DNA viruses and RNA viruses, including single-stranded, double-stranded, and partly double-stranded viruses. Certain types of viruses are enveloped viruses, meaning they are encapsulated with a lipid membrane, which comes from an infected cell when new virus particles “bud off” from the infected cell. The lipid membrane comprises material from the infected cell's plasma membrane.

With respect to RNA viruses, positive single-stranded RNA virus families include non-enveloped viruses, such as, but not limited to Astroviridae, Caliciviridae and Picornaviridae; and enveloped viruses, such as Coronaviridae, Flaviviridae, Retroviridae and Togaviridae. Negative single-stranded RNA families include but are not limited to Arenaviridae, Bunyaviridae, Filoviridae, Orthomyxoviridae, Paramyxoviridae, and Rhabdoviridae, all of which are enveloped viruses. Some embodiments of the invention include applying lectin-fingerprinting methods to RNA viruses. In certain embodiments of the invention, methods of the invention are applied to an infection by a positive single-stranded RNA virus, optionally a coronaviridae infection. In some embodiments of the invention, a virus that infects a subject is a SARS-CoV virus, and optionally is a SARS-CoV-2 virus. With respect to DNA viruses, double-stranded DNA virus families include non-enveloped viruses, which include but are not limited to: Adenoviridae, Papovaviridae, and Poxviridae, and enveloped viruses such as Herpesviridae. Single-stranded DNA virus families include non-enveloped viruses, such as but not limited to: Parvoviridae and Anelloviridae. In some embodiments of the invention, compositions and methods of the invention are applied to DNA viruses.

As used herein, the term “viral particle” refers to an infectious viral particle or virion, whose main function is to deliver its genome (DNA or RNA) into a host cell so that its genome can be expressed, e.g., transcribed and translated, by the host cell. A complete viral particle includes one or more types of viral proteins. Several different types of viral proteins exist, including structural proteins, non-structural proteins, and regulatory and accessory proteins. Non-limiting examples of viral proteins are: nucleocapsid (N) proteins and spike (S) proteins. Spike protein (S-protein) functions to aid in infecting the host subject. Spike proteins mediate attachment between the virus and the host subject's cell surface receptors, an interaction that facilitates viral entry into the host subject's cell by assisting in the fusion of the viral and host cell membranes. In some embodiments of methods of the invention a virus spike protein is a full-length spike protein (FL-Spike protein). In certain embodiments of the invention, a virus spike protein is a portion of a FL-spike protein, wherein the portion is sufficient to permit assessment of binding of preselected lectin molecules to the spike protein and identification of the neutralization status of an anti-virus antibody directed against the virus. Thus, if the length of the amino acid sequence of a FL-spike protein is n, a portion of FL-spike protein that may be used in a method of the invention may be a length that is: n minus 1, 2, 3, 4, 5, 6, 7, 8, 9, 10, 11, 12, 13, 14, 15, 16, 17, 18, 19 or more amino acids. In some embodiments, the portion of the FL-spike protein comprises at least the receptor-binding domain (RBD) sequence of the spike protein.

A viral infection in a subject may be symptomatic or asymptomatic. A symptomatic viral infection may result in clinical symptoms in a subject infected with the virus that may be detected and assessed using an embodiment of a method of the invention. Non-limiting examples of clinical symptoms include, but are not limited to, fever, shortness of breath, difficulty breathing, loss of sense of taste and/or smell, low blood oxygenation saturation, chills, vomiting, diarrhea, headache, muscle aches/pain, weakness, loss of appetite, malaise, nasal congestion, body aches, cough, sore throat, runny nose, and sneezing. It will be understood that presence, absence, and/or severity of one or more symptoms of a viral infection may be determined and/or assessed in an infected subject. Severity of a viral infection varies with different viruses and in different subjects. For example, a first subject with a viral infection may exhibit one or more symptoms such as, fever, chills, cough, etc. and a second subject with a more severe infection with the virus may exhibit some or all of the symptoms of the first subject, and also one or more of symptoms such as but not limited to trouble breathing, confusion, inability to stay awake, bluish lips or face, pain or pressure in chest, and significantly low blood oxygen saturation. It will be understood that clinical symptoms in a subject with a viral infection can be assessed and the symptoms identified by a health-care professional.

### Anti-Virus Antibodies

An essential property of a virus is infectivity, which requires intact and functional viral proteins and an intact, functional genome that can be replicated by a host cell. As used herein, “infectivity” refers to the ability of a first virus to successfully infect a host cell, which involves successful completion of multiple steps. As used herein, the term neutralizing used in reference to an anti-virus antibody means an anti-virus antibody that interferes with and/or blocks one or more aspects of a virus' ability to infect a host cell. It has now been determined that methods of the invention can be used to identify anti-virus antibodies that inhibit infectivity of a virus.

Methods of the invention include use of preselected lectin molecules to determine a status of an anti-virus antibody with respect to its ability to neutralize the virus against which the antibody is directed. As used herein, the term “directed” in reference to viral proteins and antibodies, means the antibody recognizes and binds the viral protein. For example, an anti-SARS-CoV-2 antibody may be generated to bind a SARS-CoV-2 virion protein. In such an instance, the antibody is referred to as “directed” against the SARS-CoV-2 virus. It will be understood that a subject infected with a virus may generate antibodies directed to that virus. Anti-virus antibodies may bind a virion protein of the virus they are directed against, but some anti-virus antibodies are more effective than others as therapeutic anti-virus antibodies.

Certain antibodies directed against a virus bind to a protein of the virus and can reduce a function of the virus, a non-limiting example of a virus function is entry of the virus particle into a host cell. Thus, antibodies that can reduce or eliminating entry of a virus into a host cell may be useful to treat the viral infection. Such anti-viral antibodies are referred to herein as “neutralizing” antibodies. In addition to anti-virus antibodies that are directed against a virus and can neutralize the virus, certain anti-virus antibodies may bind the virus protein they are directed against without reducing viral function and thus may not be useful to treat the viral infection. Such anti-virus antibodies are referred to herein as non-neutralizing antibodies.

Certain embodiments of methods of the invention can be used to identify a neutralizing potential of an anti-virus antibody for the virus against with is the antibody is directed. This information can be used to assess neutralization potential of a novel anti-virus antibody and to assess neutralization potential of an existing anti-virus antibody. In each instance, a method of the invention can be used to assess and determine whether a candidate anti-virus antibody is a neutralizing antibody. In some embodiments of methods of the invention, an anti-virus antibody is a purified antibody and in certain embodiments of methods of the invention an antibody is in a mixed environment, for example, though not intended to be limiting, in some embodiments, an antibody is included in a mixture comprising one or both of blood and serum.

It is known in the art that viruses are capable of mutating during replication in a host cell, and a resulting viral mutation, may result in a new, variant virus, which may be referred to herein as viral variant, virus variant, and/or infective functional variant. A viral variant may differ from its parent virus (for example the virus of which it is a variant) in a manner that an anti-virus antibody that is neutralizing with respect to the parent virus, may not be neutralizing with respect to the viral variant. Thus, mutations to a first virus can result in resistance to anti-viral therapies comprising anti-virus antibodies that were effective against the first virus. Certain embodiments of methods of the invention can be used to assess the ability of an existing anti-virus antibody to neutralize a viral variant. Using embodiments of methods of the invention, one can assess and track efficacy of a therapeutic comprising an anti-virus antibody against a mutated variant of a first virus. As used herein the term “parent virus” may be used to indicate a first virus from which a second virus, also referred to herein as a mutated first virus, virus variant, and/or viral variant arises. Aspects of the invention, in part, include methods of detecting efficacy of an anti-virus antibody against a mutated first virus. In non-limiting examples, a parent virus is a SARS-CoV virus or a SARS-CoV-2 virus and a viral variant that can be assessed using a method of the invention is a mutated SARS-CoV virus, mutated SARS-CoV-2 virus.

### Glycan-Binding Agents

Aspects of the invention comprise using one or a plurality of glycan-binding agents to identify neutralization status of an antibody. In some embodiments of methods and assays of the invention, a glycan-binding agent is a lectin. In certain embodiments a glycan-binding agent is a protein. A glycan-binding agent used in a method or assay of the invention may be a naturally occurring glycan-binding agent, a variant of a glycan-binding agent, a functional fragment of a glycan-binding agent, a recombinant glycan-binding agent, a functional fragment of a recombinant glycan-binding agent, an engineered glycan-binding agent, or an engineered fragment of a glycan-binding agent. One or more characteristics of binding of one or a plurality of glycan-binding agents to an antibody can be assessed using embodiments of methods of the invention. In some embodiments, methods of the invention are used to assess binding of a glycan-binding agent to one or more proteins of an antibody and/or one or more locations or regions of an antibody. A non-limiting example of a protein, location, or region of an antibody is a spike protein of the antibody. In some embodiments, a glycan-binding agent is a glycan-binding protein, non-limiting examples which include an antibody, an antibody fragment, and a nanobody that binds specific glycan epitopes. Additional non-limiting examples of antibodies that can be used as glycan-binding agents in a method or assay of the invention are antibodies that bind sugars and originate in organisms such as but not limited to llamas and lampreys or are designed based on antibodies produced by organisms such as but not limited to llamas and lampreys. In some embodiments of methods and assays of the invention, a glycan-binding agent comprises a glycan-binding functional fragment of an antibody produced by an organism, such as but not limited to a llama or a lamprey. As used herein, the term glycan-binding functional fragment of an antibody means a fragment of a glycan-binding antibody that comprises sufficient structure to function in an assay or method of the invention.

In some embodiments of methods and assays of the invention, a glycan-binding agent is a lectin. Non-limiting examples of lectins that may be selected and used in a method of the invention are disclosed herein. One or more characteristics of binding of one or a plurality of lectins to an antibody can be assessed using embodiments of methods of the invention. In some embodiments methods of the invention are used to assess lectin binding to one or more proteins of an antibody and/or one or more locations or regions of an antibody. A non-limiting example of a protein, location, and/or region of an antibody is a spike protein of the antibody. In some embodiments, a lectin is a complete lectin molecule and in certain embodiments a lectin molecule may be a complete lectin molecule but with glycans removed; and in certain embodiments a lectin molecule is a fragment of lectin molecule that comprises sufficient structure to function in an assay or method of the invention.

Certain embodiments of methods of the invention, may comprise one or more lectins in a binding assay and method of the invention is selected from the lectins disclosed herein. It will be understood that other art-known lectins or prepared lectins may be used certain embodiments of methods of the invention disclosed herein. In some embodiments of the invention, a lectin used to assess a virus-neutralization status of an anti-viral antibody is a lectin identified and/or isolated from a natural source. In certain embodiments of the invention, a lectin used to assess a virus neutralization status of an anti-viral antibody is a recombinant lectin. In some embodiments of methods and assays of the invention, one or more of the lectins is contacted with an enzyme to remove glycans from the lectins. In some embodiments of methods and assays of the invention, one or more lectins used to assess a virus-neutralization status of an anti-viral antibody is selected from one or more of: (1) lectins disclosed herein; (2) other art-known lectins; (3) recombinant or engineered lectin molecules; and (4) lectins treated with an enzyme or other means to remove glycans from the lectin.

Methods of the invention include use of glycan-binding agents, a non-limiting example of which are lectin molecules, which are contacted with and bind with a virus protein(s) that has been contacted with an anti-virus antibody directed against the virus. In some embodiments, the previously contacted virus/virion protein is contacted with one or with a plurality of preselected glycan-binding agents, a non-limiting example of which are preselected lectin molecules. As used herein “plurality” means more than one, for example: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more. The terms “contact” and “contacted with” as used with respect to glycan-binding agents, lectins, and virion proteins, means the glycan-binding agents and virion proteins, or lectin molecules and virion proteins, are placed in an environment in which they are capable of being in physical contact with each other and are capable of binding with each other. In some embodiments, preselected glycan-binding agents and virion proteins are in a solution with each other. In some embodiments, the solution comprising the preselected glycan-binding agents and virion proteins comprises additional components. In some embodiments, preselected lectins and virion proteins are in a solution with each other, and the solution may include other components. In some embodiments, the solution comprising the preselected lectins and virion proteins comprises additional components.

Non-limiting examples of procedures that can be used in methods of the invention are referred to herein as Lectin Fingerprinting enzyme-linked lectin assay (ELLA). In certain embodiments of an ELLA assay used in a method of the invention, FL-Spike and RBD are in solution and plated in individual containers, a non-limiting example of which are wells in a Nunc MaxiSorp™ plate. The plate is sealed and left to absorb overnight at 4° C., mixed with a solution of pre-selected detectably labeled lectin molecules, and after a period of binding the plate contents are read to determine binding between the lectin molecules and the virus protein (see Examples for more complete description of an embodiment of the ELLA method).

Certain aspects of the in invention include methods in which a competitive ELLA is performed. In certain of these methods, FL-Spike and RBD are in solution and plated in individual containers, a non-limiting example of which are wells in a Nunc MaxiSorp™ plate. The plate is sealed and left to absorb overnight at 4° C., mixed with a solution of pre-selected detectably labeled lectin molecules, and after a period of binding, anti-virus antibodies are added to the mixtures. When reactions are complete the plates are read to determine binding between the lectin molecules and the virus protein, which identifies competition between the anti-virus antibody bound to the viral protein and one or more of the preselected lectin molecules (see Examples for more complete description of an embodiment of the competitive ELLA method).

An additional, non-limiting method that may be included in certain embodiments of a method or assay of the invention is a high-throughput method of detecting one or more characteristics of binding between one or more preselected glycan-binding agents and a virion protein. Another non-limiting method that may be included in certain embodiments of a method or assay of the invention is a high-throughput method of detecting one or more characteristics of binding between one or more lectins with a virion protein. High throughput methods are known in the art and a practitioner will be able to use such methods in conjunction with details disclosed herein to identify a neutralization status of an anti-virus antibody.

Lectins that may be used in certain embodiments of methods of the invention include, but are not limited to insect lectin molecules, plant lectin molecules, and mammalian lectin molecules. Preselected lectin molecules used in certain embodiments of the invention are one or more of Concanavalin A (ConA), Dolichos biflorus Agglutinin (DBA), Ricinus communis Agglutinin I (RCA I, also referred to as RCA120), Peanut Agglutinin (PNA), Soybean Agglutinin (SBA), Ulex europaeus Agglutinin I (UEA I); Wheat Germ Agglutinin (WGA), Griffonia simplicifolia Lectin I (GSL I), Lens culinaris Agglutinin (LCA), Phaseoius vulgaris Erythroagglutinin (PHA-E), Phaseoius vulgaris Leucoagglutinin (PHA-L), Pisum sativum Agglutinin (PSA), Lotus tetragonolobus Lectin (LTL), Datura stramonium Lectin (DSL), Erythrina cristagalli Lectin (ECL), Griffonia (Bandeiraea) Simplicifolia Lectin II (GSL II), Jacalin, Lycopersicon esclentum (Tomato) lectin (LEL), Solanum tuberosum (Potato) Lectin (STL), Vicia villosa Lectin (VVL), Aleuria aurantia Lectin (AAL), Maackia amurensis Lectin I (MAL I), Maackia amurensis Lectin II (MAL II), Sambucus negra Lectin (SNL), RPL-αGal, RPL-Gal1, RPL-αMan, RPL-Man2, RPL-Sia1, and RPL-Fuc1.

The term “preselected” as used herein with respect to lectin molecules means chosen for inclusion. Preselection may be based, at least in part, on the specific virus the anti-viral antibody is directed against. For example, a preselected lectin molecule chosen and used in a method of the invention to identify the neutralization status of an anti-SARS-CoV-2 virus antibody may be different than a preselected lectin molecule chosen and used to identify the neutralization status of an anti-virus antibody that recognizes and was raised against a different virus, for example a Rhabdoviridae virus.

### Virus Neutralization

Certain embodiments of methods of the invention include identifying a virus-neutralization status of an anti-virus antibody. As used herein the “status” of virus neutralization can be determined to be “present” or “absent”. The virus-neutralization status of an anti-virus antibody is present if, when contacted with the virus, the anti-virus antibody can neutralize the virus. The virus-neutralization status of an anti-virus antibody is absent if, when contacted with the virus, the anti-virus antibody is not capable of neutralizing the virus. In some embodiments of the invention, a status of virus-neutralization that is determined to be present can include assessing strength of the virus neutralization compared to other virus-neutralizing antibodies. For example, a status of a virus-neutralizing antibody can be determined to be “strongly neutralizing” or “weakly neutralizing” as compared to other virus-neutralizing antibodies. Non-limiting examples of neutralizing, strongly neutralizing, weakly neutralizing, and non-neutralizing anti-virus antibodies are described herein.

Some embodiments of methods of the invention with which to identify a virus-neutralization status of an anti-virus antibody, include contacting a viral particle, also referred to herein as a “virion” of the virus, which has previously been in contact with an anti-virus antibody, with one or more preselected lectin molecules. A virion that is contact with an anti-virus antibody directed against the virus will exhibit binding between the anti-virus antibody and a protein of the virion, for example a virion spike protein. This binding results the inaccessibility of the virion protein to binding by lectins. Certain embodiments of methods of the invention, include contacting a virion protein that has previously been contacted with the anti-virus antibody with one or a plurality of lectins and determining one or more characteristics of binding between the preselected lectin molecule(s) and the virion protein. The determined binding characteristic(s) may be used to identify a virus-neutralization status of the anti-virus antibody.

In some embodiments of methods of the invention, determined binding characteristics include characteristic such as, but not limited to: the presence or absence of binding one or more of the preselected lectin molecule(s) with the virion protein; a level or amount of binding of one or more of the preselected lectin molecule(s) with the virion protein; location(s) of the binding of the one or more preselected lectin molecule(s) on the virion protein; the identity of the preselected lectin molecule(s) that bind the virion protein; and the identity of the preselected lectin molecule(s) that do not bind the virion protein. Thus, methods of the invention may include determining one or more binding characteristics such as presence or absence, a level (amount) of binding, location of binding, identity of preselected lectins that do bind and lectins that don't bind when contacted with a virion that was previously contacted with an anti-virus antibody with the results identifying the neutralization status of the anti-viral antibody.

In some embodiments, a method of the invention comprises first contacting a virion particle with one or a plurality of selected lectins. When the lectin(s) contact the virion particle, one or more of the lectins bind the virion particle forming a lectin/virion complex. A lectin/virion complex comprises one or more of the lectins bound to the virion particle, which may be referred to herein as lectin(s) bound to a protein of the virion particle. After contacting the virion particle with one or a plurality of selected lectins, the lectin/virion complexes are then exposed to one or more antibodies. The term “exposed” when used in reference to lectin/virion complexes and antibodies, means the lectin/virion complexes are contacted with the antibodies. Methods and assays of the invention comprise detecting whether one or more lectins bound to the virion are displaced as a result of the antibody. Methods of the invention comprise determining one or more binding characteristics of the preselected lectin molecule(s) with the virion particle/protein of the antibody-contacted virion. Methods and assays of the invention can be used to determine binding characteristics of the lectin and virion particle. Non-limiting examples of characteristics that may be determined are presence of binding; absence of binding, level (amount) of binding, location of binding on the virion particle, identity of a lectin that binds to the virion particle, and identity of a lectin that does not bind the virion particle. Displacement of one or more bound lectins identifies binding characteristics of the lectin(s) with the virion and can be used to identify virus-neutralization status of the antibody.

In a non-limiting example, a plurality of preselected lectin molecules comprising each of ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1 is contacted with a sample containing a virion and the contacted sample is subsequently contacted with an anti-virus antibody from serum of a convalescent subject who has had SARS-CoV-2. The presence or absence of binding of a protein of the virion with one or more of the preselected lectin molecules is determined and results indicate presence of binding of the virion protein with ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1. This result identifies the anti-virus antibody as a SARS-CoV-2-neutralizing antibody.

In another non-limiting example, a plurality of preselected lectin molecules comprising MALI, RPL-αMan, RPL-Man2, PNA, RPL-Gal1, Jacalin, RPL-αGal, RPL-Sia1, RPL-Fuc1, DBA, and LTL is contacted with a sample containing a virion and then the contacted sample is further contacted with a purified anti-virus antibody raised against SARS-CoV-2 virus. The presence or absence of binding of a protein of the virion with one or more of the preselected lectin molecules is determined, and the results indicate presence of binding of the virion protein with MALI, RPL-αMan, RPL-Man2, PNA, RPL-Gal1, Jacalin, RPL-αGal, RPL-Sia1, RPL-Fuc1, DBA, and LTL. This result identifies the anti-virus antibody as a SARS-CoV-2-neutralizing antibody.

In some embodiments, methods of the invention are used to identify the status of a virus-neutralizing anti-virus antibody in convalescent sera obtained from a subject who had or has a viral infection. Convalescent sera are sera obtained from a subject who has, or previous had, a viral infection by the virus for which an anti-virus antibody's virus neutralization status is being determined. An anti-virus antibody present in convalescent sera is an antibody that has been generated in the subject that has been infected with the virus. In some embodiments of the invention, a serum sample is obtained from a subject who has, or has had, a viral infection and one or more anti-virus antibodies in the serum sample are assessed using a method of the invention and the status of virus neutralization is determined for one or more anti-virus antibodies in the convalescent.

In some embodiments, methods of the invention are used to identify the status of a virus-neutralizing anti-virus antibody in sera obtained from a subject who has not had the viral infection but has been vaccinated against the virus. An anti-virus antibody present in sera from a subject that has not had the virus but has received a vaccination for the virus is an antibody that has been generated in the subject not infected by the virus. In some embodiments of the invention, a serum sample is obtained from a subject who has not had the virus, a non-limiting example of which is a CoV-2 virus and has received one or more vaccinations with a CoV-2 vaccine and one or more anti-virus antibodies in the serum sample from the subject are assessed using a method of the invention and the status of virus neutralization is determined for one or more anti-virus antibodies generated in the subject. In some embodiments, the subject has received one vaccination against the virus and in certain embodiments of methods of the invention, the subject has received two or more vaccinations against the virus. In some embodiments, the same type of vaccine was administered each time a subject received two or more vaccinations against the virus. In some embodiments, a subject that has received two or more vaccinations may have received two or more different doses of the same vaccine. In certain embodiments, two or more different types of vaccines were administered to a subject that received two or more vaccinations against the virus. It will be understood that the term “different type” as used herein in reference to a vaccine means one or more of: vaccines produced using different viral components or other aspect that distinguishes the vaccines and their immunological effect in a subject. Non-limiting examples of different vaccines are vaccines developed by different manufacturers.

In some embodiments, methods of the invention are used to identify the status of a virus-neutralizing anti-virus antibody in sera obtained from a subject who has had the viral infection and has been vaccinated against the virus. In some embodiments, the subject was vaccinated prior to having the virus; in some embodiments, the subject was vaccinated subsequent to having the virus, and in some embodiments the subject was vaccinated before and subsequent to having the virus. In some embodiments the subject has been vaccinated with two or more different vaccines against the virus.

Another type of anti-virus antibody that can be assessed using certain embodiments of the invention is a referred to herein as a purified anti-virus antibody. Unlike an antibody in a serum sample obtained from a subject, which may include more than one different anti-virus antibodies, a purified antibody may be a manufactured antibody, a recombinant antibody, a humanized antibody, a synthetic antibody, an engineered antibody, a monoclonal antibody, a purified polyclonal antibody, and/or a functional fragment of any of the aforementioned types of purified antibodies. In some embodiments, an anti-virus antibody is generated in a subject, collected in a blood sample, and separated from all other, different antibodies in the blood sample, thus resulting in a purified anti-virus antibody obtained from the convalescent serum sample.

Some aspects of the invention comprise competitive fingerprinting methods and assays to identify therapeutic efficacy of peptide binders. For example, in some embodiments of methods and assays of the invention include contacting sera from a subject with lectins to produce virion/lectin complexes and then contacting the complexes with candidate non-antibody agents that can inhibit the virus. Determination of the presence or absence of an effect on the lectin binding resulting from the contact with the non-antibody candidate agent provides a measure of the efficacy of the candidate agent against the virus. In some embodiments, a non-antibody candidate agent that is used in such methods and assays of the invention is a molecule that binds the virion particle or is being tested to determine whether it binds the virion particle. A non-limiting example of a non-antibody candidate agent that may be used in methods and assays of the invention is a peptide.

Methods and assays of the invention can be used to identify proteins, peptides, or small molecules capable of blocking viral entry into a cell. In some embodiments, proteins, peptides, or small molecules tested using methods and assays of the invention proteins, peptides, or small molecules, respectively that have been engineered to block viral entry. In some embodiments of methods and assays of the invention. Candidate proteins, peptides, and/or small molecules can be tested in mixtures with antibodies using methods and assays of the invention.

### Treatments

Methods of the invention, in some embodiments may be used to identify a neutralizing anti-virus antibody, and additionally to select and/or administer the identified neutralizing anti-virus antibody to a subject in need of such treatment as part of a therapeutic regimen to treat an infection of the subject by the virus. It will be understood that practicing certain embodiments of a method of the invention may identify that an anti-virus antibody is a non-neutralizing anti-virus antibody, and the method may also include selecting a therapeutic regimen for the subject that does not include administering the identified non-neutralizing anti-virus antibody as a treatment for an infection of the subject by the virus.

As used herein the terms “treat”, “treatment”, and “treating” used in reference to a viral infection mean one or more of: preventing onset of the viral infection in a subject, preventing progression of the viral infection in a subject, reducing progression of the viral infection in a subject, reducing the viral infection in a subject, reducing severity of the viral infection in a subject, reducing symptoms of the viral infection in a subject, preventing spread of the viral infection from a subject to one or more additional subjects, etc. It will be understood that treating or treatment of a subject using an anti-virus antibody, can but need not totally eliminate or prevent the subject from having an infection by the virus viral infection in a subject who generates or who has been administered the anti-virus antibody. Examples a viral function that may be reduced and/or eliminated by certain anti-virus antibodies include but are not limited to virus replication, virus release, virus infectivity, etc. An anti-virus antibody that is effective at reducing a viral infection may be referred to herein as a neutralizing anti-virus antibody for the virus it is directed against. The term “block” and “blocking” as used herein in reference to inhibiting activity of a virus, means reducing activity of the virus. For example, blocking infection by the virus using a method of the invention, may reduce viral infection in a cell and/or subject. It will be understood that blocking infection by a virus in a cell or subject using a composition and/or method of the invention, can but need not totally eliminate or prevent the viral infection the subject. Some embodiments of methods of the invention comprise blocking infection of a cell and/or subject by a virus wherein the viral infection in the cell or subject is at least 0.5%, 1%, 2%, 3%, 4%, 5%, 6%, 7%, 8%, 9%, 1%, 15%, 20%, 25%0, 30%, 35%, 40%, 45%, 50%, 55%, 60%, 65%, 70%, 75%, 80%, 85%0, 90%, 95%, 98%, 99%, or 100% inhibited compared to the viral infection in a control cell and/or subject.

An effective treatment of viral infection in a subject by administering to a subject an identified neutralizing anti-virus antibody may result in one or more symptoms of the viral infection in the subject that is statistically significantly ameliorated in comparison to the one or more symptoms in a subject not administered the neutralizing anti-virus antibody, or administering a different amount of the neutralizing anti-virus antibody—e.g. in comparison to the one or more symptoms resulting from a control treatment. A statistically significant amelioration of one or more symptoms of a viral infection in a subject may be an at least 0.5%, 1%, 2%, 3%, 4%, 5%, 6%, 7%, 8%, 9%, 10, 15%, 20%, 25%, 30%, 35%, 40%, 45%, 50%, 55%, 60%, 65%, 70%, 75%, 80%, 85%, 90%, 95%, 98%, 99%, or 100% reduction compared to a control treatment, including all percentages within that range. As used herein, the term “amelioration” refers to improvement in severity of one or more symptoms of a viral infection in a cell or subject compared to a control treatment or compared to the severity of one or more symptoms as determined at an earlier time point in the viral infection. Non-limiting examples of amelioration also include a reduction in number and/or severity of one or more symptoms of the viral infection in an infected subject, and a reduction in overall duration of symptoms in an infected subject, and a reduction in viral load in a subject. Amelioration of viral infection symptoms may be evaluated and/or measured using art-known methods that will be familiar to those with ordinary skill in the art. In some aspects, treating a viral infection also refers to treating a viral infection such that symptoms of an infection in a subject are eliminated or apparently eliminated compared to symptoms previously exhibited by a subject, though it will be understood that in other aspects, one or more symptoms of a viral infection may persist in a subject despite the viral infection having been reduced or lessened.

### Labelling

In certain aspects of the invention, a lectin molecule may comprise a detectable label, thereby enabling visual identification and localization of the lectin molecule. In some embodiments, the presence of a detectable label can be used to help identify if a particular preselected lectin molecule is bound to a virion protein or is not bound to a virion protein with which it has been contacted. As used herein, a detectable label may be physical part of or is chemically bound (e.g., covalently, or via hydrogen or ionic bonding) to the lectin molecule and can be detected through microscopy or other means of detection. In some embodiments, the detectable label comprises a biotin label, a fluorescent label, a luminescent label, a radiolabel, an enzymatic label, a contrast agent, a heavy metal, or a heavy element such as bromine or iodine, or metals such as gold, osmium, rhenium, etc. In some embodiments, the detectable label is a fluorescent protein or other light-emitting protein. Numerous detectable labels are well known in the art and routine methods can be used to include such labels in lectin molecules and in methods of the invention. In some embodiments, a lectin molecule may include more than one detectable label.

### Cells, Samples, and Subjects

It will be understood that a cell in a subject may be referred to herein as a subject's cell or a host subject cell or a host cell. A sample used in a method of the invention may be referred to as a biological sample. A biological sample may include one or more of a cell, an anti-virus antibody, a virion, a virion that has been contacted with an anti-virus antibody, etc. A biological sample may be prepared in a laboratory and/or may be withdrawn, cut, collected, and/or obtained from a subject. Non-limiting examples of biological samples that may be used in certain embodiments of methods of the invention are serum, tears, urine, saliva, cerebrospinal fluid, lymph, or another bodily material that is believed may comprise virus proteins that have been previously contacted with an anti-virus antibody.

In some embodiments of the invention, a subject is known or suspected to currently have, or to previously have had a viral infection for which a neutralization status of an anti-virus antibody is being assessed. It will be understood that a sample for testing in a method of the invention may, in some embodiments, be a sample obtained from culture.

As used herein, the term “subject” may refer to human or non-human animals, including mammals and non-mammals, vertebrates, and invertebrates, and may also refer to any genetically modified or engineered organism. Non-limiting examples of mammalian subjects include primates (including but not limited to humans and non-human primates), rodents (including but not limited to mice, rats, squirrels, chipmunks, prairie dogs), bats, lagomorphs, deer, canids, felids, bears, horses, cows, sheep, goats, and pigs. A subject may be considered to be a normal subject or may be a subject known to have or suspected of having a viral infection, such as, but not limited to a SARS-CoV infection, additional non-limiting examples of viral infections are provided elsewhere herein.

### Controls

Certain embodiments of methods of the invention used to identify a status of an anti-virus antibody or other anti-virus agent comprise comparing results obtained for a purified antibody sample, a convalescent serum sample, and/or other subject serum sample, with a control value obtained from a control sample or plurality of control samples. A non-limiting example of a control sample is a sample comprising a known neutralizing anti-viral antibody. In some embodiments, control binding characteristics comprise binding characteristics of one or more lectins with a virion particle in the absence of an antiviral agent, a non-limiting example of which is an anti-virus antibody. In some embodiments, a control to which a candidate antiviral agent's effect on binding may be compared, comprises binding characteristics of one or more lectins with a virion particle in the absence of a candidate agent such as an antiviral antibody, a peptide binder, a protein binder, or other candidate agent to block or neutralize the virus. In some embodiments, one or more control binding characteristics are binding characteristics of an antiviral agent that is effective at blocking infection by the virus. In some embodiments, one or more control binding characteristics are binding characteristics of an antiviral agent that is effective at neutralizing the virus. In some embodiments, one or more control binding characteristics are binding characteristics of an antiviral agent that is effective at blocking infection by the virus. In some embodiments, one or more control binding characteristics are binding characteristics of an antiviral agent that is effective at neutralizing the virus. A control value for binding characteristics between one or more lectin molecules and a virion protein and/or potential neutralization can be established based upon comparative groups such as lectin binding characteristics of an antiviral agent that neutralizes the virus. Those in the art will readily identify suitable control subjects and samples for use with methods and assays of the invention.

### EXAMPLES

### Example 1

### Material and Methods

### Reagents

Biotinylated plant and fungal lectins were purchased from Vector Labs. Lectins received as lyophilized powders were resuspended at 2 mg/mL in 20 mM HEPES, 150 mM sodium chloride. Biotinylated prokaryotic lectins were provided by GlycoSeLect, Ltd. (Dublin, Ireland) (Table 1). SARS-CoV-2 FL-Spike and RBD were recombinantly expressed as previously described. (Norman, M. et al., Nat. Biomed. Eng. 2020, 1-8) Recombinant FL-Spike protein was stored in PBS at 4° C. for up to 1 week. Recombinant RBD was aliquoted and stored at −20° C.

TABLE 1
Certain materials and sources

| Lectin | Abbr | Specificity | Vendor | Cat. No. |
|---|---|---|---|---|
| Concanavalin A | ConA | Mannose, Glucose | Vector Laboratories | BK-1000 |
| Glycine max | SBA | terminal α- or β- | Vector Laboratories | BK-1000 |
| (soybean) | | linked N- | | |
| agglutinin | | acetylgalactosamine | | |
| | | and galactose | | |
| Triticum vulgaris | WGA | N-acetylglucosamine | Vector Laboratories | BK-1000 |
| (wheat germ) | | | | |
| agglutinin | | | | |
| Dolichos biflorus | DBA | α-linked N- | Vector Laboratories | BK-1000 |
| agglutinin | | acetylgalactosamine | | |
| Ulex europaeus | UEA I | α-linked fucose | Vector Laboratories | BK-1000 |
| agglutinin I | | | | |
| Ricinus | RCA120 | Galactose, Lactose | Vector Laboratories | BK-1000 |
| communis | | | | |
| agglutinin | | | | |
| Arachis | PNA | galactosyl (β-1,3) N- | Vector Laboratories | BK-1000 |
| hypogaea | | acetylgalactosamine | | |
| (peanut) | | | | |
| agglutinin | | | | |
| Griffonia | GSL I | α-N- | Vector Laboratories | BK-2100 |
| (Bandeiraea) | | acetylgalactosamine, | | |
| simplicifolia | | α-galactose | | |
| lectin | | | | |
| Pisum sativum | PSA | α-linked mannose- | Vector Laboratories | BK-2100 |
| lectin | | containing | | |
| | | oligosaccharides, | | |
| | | with an N- | | |
| | | acetylchitobiose- | | |
| | | linked α-fucose | | |
| | | residue included | | |
| Len culinaris | LCA | Mannose, Glucose | Vector Laboratories | BK-2100 |
| agglutinin | | | | |
| Phaseolus | PHA-E | Galactose, Complex | Vector Laboratories | BK-2100 |
| vulgaris | | Structures | | |
| Erythroagglutinin | | | | |
| Phaseolus | PHA-L | Galactose, Complex | Vector Laboratories | BK-2100 |
| vulgaris | | Structures | | |
| Leucoagglutinin | | | | |
| Datura | DSL | (β-1,4) linked N- | Vector Laboratories | BK-3000 |
| Stramonium | | acetylglucosamine | | |
| lectin | | oligomers, N- | | |
| | | acetyllactosamine | | |
| Erythrina | ECL | Galactose, N- | Vector Laboratories | BK-3000 |
| cristagalli lectin | | Acetylgalactosamine, | | |
| | | Lactose | | |
| Griffonia | GSL II | α- or β-linked N- | Vector Laboratories | BK-3000 |
| (Bandeiraea) | | acetylglucosamine | | |
| simplicifolia | | | | |
| lectin II | | | | |
| Jacalin | Jacalin | O-glycosidically | Vector Laboratories | BK-3000 |
| | | linked galactosyl (β- | | |
| | | 1,3) N- | | |
| | | acetylgalactosamine | | |
| Lycopersicon | LEL | [GlcNAc]1-3, N- | Vector Laboratories | BK-3000 |
| esculentum | | Acetylglucosamine | | |
| (tomato) lectin | | | | |
| Vicia villosa | VVL | α- or β-linked | Vector Laboratories | BK-3000 |
| agglutinin | | terminal N- | | |
| | | acetylgalactosamine | | |
| Aleuria Aurantia | AAL | (α -1,2) linked fucose | Vector Laboratories | B-1395-1 |
| Lectin | | residues, fucose | | |
| | | linked (α -1,6) to N- | | |
| | | acetylglucosamine or | | |
| | | to fucose linked (α - | | |
| | | 1,3) to N- | | |
| | | acetyllactosamine | | |
| | | related structures | | |
| Maackia | MAL II | sialic acid in an (α- | Vector Laboratories | B-1265-1 |
| Amurensis | | 2,3) linkage | | |
| Lectin II | | | | |
| Maackia | MAL I | gal (β-1,4) glcNAc, | Vector Laboratories | B-1315-2 |
| Amurensis | | galactose, N- | | |
| Lectin I | | acetyllactosamine | | |
| | | with sialic acid at the | | |
| | | 3 position of | | |
| | | galactose | | |
| Sambucus Nigra | SNL | sialic acid attached to | Vector Laboratories | B-1305-2 |
| Lectin | | terminal galactose in | | |
| | | α-2,6 and to a lesser | | |
| | | degree, α-2,3 | | |
| | | linkage. | | |
| Lotus | LTL | Fucose, Arabinose | Vector Laboratories | B-1325-2 |
| tetragonolobus | | | | |
| lectin | | | | |
| Solanum | STL | N-acetylglucosamine | Vector Laboratories | BK-3000 |
| Tuberosum | | | | |
| (Potato) Lectin | | | | |
| RPL-αGal | αGal | Terminal α-linked | GlycoSeLect, Ltd. | L-001 |
| | | Galactose & N- | | |
| | | Acetylgalactosamine | | |
| | | (GalNAc) | | |
| RPL-Gal1 | Gal1 | Terminal β-linked | GlycoSeLect, Ltd. | L-002 |
| | | Galactose & N- | | |
| | | Acetyllactosamine | | |
| | | (LacNAc) | | |
| RPL-αMan | αMan | Fucose/Mannose: | GlycoSeLect, Ltd. | L-006 |
| | | Lewis a (Lea), Lewis | | |
| | | x (Lex) & terminal | | |
| | | α-mannose | | |
| RPL-Man2 | Man2 | Terminal α -mannose | GlycoSeLect, Ltd. | L-007 |
| RPL-Sia1 | Sia1 | Terminal α2-3-linked | GlycoSeLect, Ltd. | L-008 |
| | | Sialic Acid | | |
| | | (Neu5Ac) - on both | | |
| | | N-linked and O- | | |
| | | Linked Glycans | | |
| RPL-Fuc1 | Fuc1 | α-linked Fucose | GlycoSeLect, Ltd. | L-011 |

### Human Subjects

A power analysis was performed to determine the number of vaccinated patient samples required for this study. Effect size was estimated to be modest (0.3-0.5) to strong (0.8) based on the perturbation of lectins by commercial neutralizing antibodies. Using these data, it was estimated that 4-16 patient samples would be required to achieve 80% power with a significance level alpha=0.05.

Pre-vaccinated patient sera were collected in compliance with IRB protocol IRB2020P000849, Biorepository for samples from those at increased risk for or infected with SARS-CoV-2. Use of vaccinated patient sera for the development and validation of SARS-CoV-2 diagnostic tests was approved by Partners Institutional Review Board (IRB2020P000895). Serum samples from 16 patients diagnosed with COVID-19 (confirmed by at least one SARS-CoV-2 PCR-positive nasopharyngeal swab at Massachusetts General Hospital) were collected over course of several weeks, resulting in partially longitudinal, cross-sectional cohort consisting of 16 serum samples. Antibodies α-S1-MM43 (Mouse mAb, 40591-MM43, Sino Biological, epitope Val16-Arg685 on the S1 portion of recombinant Spike protein), α-RBD-D002 (Chimeric mAb, 40150-D002, Sino Biological, Arg306-Phe527 on recombinant Spike RBD) and α-S1-CR3022 (ab273073-200UG, Abcam, binds the amino acids 318-510 in the S1 domain of the SARS-CoV Spike protein as well as SARS-CoV-2 (COVID-19) Spike protein) were purchased and stored according to manufacturers' recommendations. Patient antibody samples were collected in compliance with IRB protocol 2020P000895 and vaccinated patient sera were collected in compliance with IRB protocol 2020P002274.

### Cell Lines

HEK293T cells (ATCC) were cultured in DMEM (Corning) containing 10% fetal bovine serum (VWR), and penicillin/streptomycin (Corning) at 37° C. and 5% CO₂. 293T-ACE2 cells were a gift from Michael Farzan (Scripps Florida) and Nir Hacohen (Broad Institute) and were cultured under the same conditions as HEK293T cells. Confirmation of ACE2 expression in 293T-ACE2 cells was done via flow cytometry. The African Green Monkey Vero E6 with stable TMPRSS2 expression was constructed by lentiviral transduction of Vero E6 (ATCC) using the construct pTRIP-SFFV-Hygro-2A-TMPRSS2 and selection at 500 μg/ml hygromycin. Vero E6 TMPRSS2+cells were maintained in Dulbecco's Modified Eagle's Medium (DMEM, 25 mM glucose, 4 mM glutamine, 1 mM sodium pyruvate, Gibco, #11965118) supplemented with 10% fetal bovine serum (FBS), 100 U/mL penicillin/streptomycin and 250 μg/ml hygromycin at 37° C. with 5% CO₂.

### SARS-CoV-2 Lentiviral Neutralization Assay

Neutralization was determined using a SARS-CoV-2 pseudovirus expressing a luciferase reporter gene, as described previously (Yang, Z.-Y. et al., Nature 2004, 428 (6982), 561-564). To generate the pseudovirus, the packaging construct psPAX2 (Cat #11348, AIDS Reagent), luciferase reporter plasmid pLenti-CMV Puro-Luc (Cat #17447, Addgene) and Spike protein expressing pcDNA3.1-SARS CoV-2.SΔCT were transfected in HEK293T cells by the calcium phosphate method at a ratio of 1:1:0.5. Supernatants were collected and filtered with a 0.45-μm filter 48 hours post-transfection. For the neutralization assay, HEK293 Ts were transfected with pcDNA3.1(−)-hACE2 (Cat #1786, Addgene). The hACE2-expressing HEK293T cells were plated in 96-well plates 12 hours after transfection at a density of 20,000 cells/well and rested overnight. Serum was heat inactivated by incubation at 56° C. for 30 minutes. Heat inactivated serum was twofold serially diluted, mixed with 50 μL of pseudovirus, and incubated at 37° C. incubator for 1 hour. After incubation, the serum/pseudovirus mixed was added to the HEK293T/hACE2 cells. Six hours after infection, cell medium was replenished. Cells were lysed in Steady-Glo Luciferase Assay (Promega) 48 hours after infection. A luciferase assay was performed with luciferase assay reagent (Promega) according to the manufacturer's protocol. NT50 was defined as the concentration of serum required to achieve half-maximal neutralization. (GBD 2016 Lower Respiratory Infections Collaborators, Lancet Infect. Dis. 2018, 18 (11), 1191-1210)

### Authentic SARS-CoV-2 Neutralization Assay

To evaluate neutralization activity of serum samples against authentic SARS-CoV-2, Vero E6-TMPRSS2 were seeded at 10,000 cells per well in CellCarrier-384 ultra microplate (Perkin Elmer) the day prior to infection. Patient serum samples were tested at a starting dilution of 1:40 and were serially diluted 4-fold up to four dilution spots. Serial diluted patient sera were mixed separately with diluted SARS-CoV-2 virus and incubated at 37° C. with 5% CO₂ for 1 hour. Sera-virus complexes were added to the cells. Plates were incubated at 37° C. with 5% CO₂ for 48 hours. Cells were fixed and inactivated using 4% paraformaldehyde in PBS for 2 hours at room temperature. Plates were then washed and incubated with diluted anti-SARS-CoV/SARS-CoV-2 nucleoprotein mouse antibody (Sino) for 1.5 hours at room temperature. Plates were subsequently incubated with Alexa488-conjugated goat anti-mouse (JacksonImmuno) for 45 mins at room temperature, followed by nuclear staining with Hoechst 33342 (ThermoFisher). The fluorescence images were recorded and analyzed using Opera Phenix™ High Content Screening System. The half-maximal inhibitory dilutions (ID₅₀) were determined using four parameters nonlinear curve fitting algorithm.

### Lectin Fingerprinting Enzyme Linked Lectin Assay (ELLA)

FL-Spike and RBD were diluted in PBS pH 7.5 to a final concentration of 10 μg/mL. 50 μL FL-Spike or RBD solution was plated onto 96 well Nunc MaxiSorp™ plates, sealed, and left to adsorb overnight at 4° C. Plates were blocked with 5% BSA in PBS containing 0.1% Tween-20. Blocking occurred for 2 h, r.t. or overnight at 4° C. Biotinylated lectins were diluted to 1 μg/mL in 20 mM HEPES pH 7.4, 150 mM sodium chloride, 10 mM calcium chloride, 0.1% Tween-20, 0.1% BSA. For dose dependence studies, lectins were added at 1, 10, 100 μg/mL. Lectins were added in duplicate (50 μL per well) and allowed to bind for 2 h, r.t., with agitation. Plates were washed three times with PBS containing 0.1% Tween 20 (PBST). Lectins were visualized with StrepTactin-HRP conjugate (BioRad, cat. no. 1610381; 1:10,000 dilution in 20 mM HEPES pH 7.4, 150 mM NaCl, 10 mM CaCl₂, 0.1% BSA, 0.1% Tween-20), 1 hour, r.t. with agitation. Plates were washed three times with PBST. Each well received 50 μL of 1-Step Ultra TMB-ELISA substrate solution (Thermo Fisher cat. 34028), and the reactions were quenched with 50 μL of 1 M sulfuric acid. Time of TMB-ELISA incubation was equal across wells, and wells were quenched in same order of addition. Plates were read at 450 nm. Assays were run in duplicate across experiments.

### Competitive ELLA

FL-Spike, WT RBD, and Delta RBD were diluted in PBS pH 7.5 to a final concentration of 10 μg/mL. 50 μL FL-Spike or RBD solution was plated onto 96 well Nunc MaxiSorp™ plates, sealed, and left to adsorb overnight at 4° C. Plates were blocked with 5% BSA in PBS containing 0.1% Tween-20. Blocking occurred for 2 h, r.t. or overnight at 4° C. Biotinylated lectins were diluted to 1 μg/mL in 20 mM HEPES pH 7.4, 150 mM NaCl, 10 mM CaCl₂), 0.1% Tween-20, 0.1% BSA. Lectins were added in duplicate (50 μL per well) and allowed to bind for 2 h, r.t., with agitation. Plates were washed three times with PBS containing 0.1% Tween 20 (PBST). 50 μL of antibodies (1 μg/mL in 20 mM HEPES pH 7.4, 150 mM NaCl, 10 mM CaCl₂), 0.1% BSA) or sera (1:100 dilution in 20 mM HEPES pH 7.4, 150 mM NaCl, 10 mM CaCl₂), 0.1% BSA) were added to corresponding wells and allowed to bind for 1 hour, r.t., with agitation. Plates were washed three times with PBST. Lectins were visualized with StrepTactin-HRP conjugate (BioRad, cat. no. 1610381; 1:10,000 dilution in 20 mM HEPES pH 7.4, 150 mM NaCl, 10 mM CaCl₂, 0.1% BSA, 0.1% Tween-20), 1 hour, r.t. with agitation. Plates were washed three times with PBST. Each well received 50 μL of 1-Step Ultra TMB-ELISA substrate solution (Thermo Fisher cat. 34028), and the reactions were quenched with 50 μL 1 M sulfuric acid. Time of TMB-ELISA incubation was equal across wells, and wells were quenched in same order of addition. Plates were read at 450 nm.

### PNGase Treatment

1 μg WT RBD, Delta RBD and FL-Spike were subjected to PNGase F (NEB, P0704S) treatment under native and denaturing conditions, according to the manufacturer's protocol. Briefly, 1 μg RBD and FL-Spike were each combined with 2 μL 10× glycobuffer, 2 μL 10% NP-40, and water to 20 μL. 2 μL of PNGase F was added and incubated overnight at 37° C. to yield deglyosylated native proteins. For denaturing protocol, 1 μg RBD and FL-Spike™ were combined with 1 μL 10× denaturing glycobuffer and water up to 10 μL final volume. Proteins were denatured at 95° C. for 10 minutes and then cooled on ice briefly. 1 μL PNGase was added to the denatured proteins and incubated for 1 hour at 37° C. Proteins were combined with gel loading buffer and run on SDS-PAGE (Bio-Rad, 4-15% Mini-Protean™ TGX Stain free gel, cat. 4568086). Proteins were visualized on Chemi-Doc™ MP system (Bio-Rad) after excitation with UV to activate stain-free reaction.

### Comparative Glycoproteomics of WT and Delta Spike RBD

50 μg WT and Delta RBD were dissolved in 25 μL digestion buffer (50 mM Ammonium Bicarbonate) and homogenized. 25 μL of 25 mM dithiothreitol (DTT) was added and incubated at 45° C. for 45 mins, followed by 25 μL of 90 mM iodoacetamide and incubated at room temp in the dark for 20 mins. Following incubation, samples were desalted using 10 kDa Amicon Ultra centrifuge filters (cat. UFC501096). The sample was then digested with Trypsin (1:20 E:S) at 37° C. overnight. The reaction was stopped by heating samples to 100° C. for 5 min. The sample was then de-N-glycosylated using 2 μL of PNGase F (NEB) and incubated at 37° C. overnight. The samples were then loaded onto a C18 SPE cartridge (Resprep cat. 26030), and the released N-glycans were eluted with 5% acetic acid. The de-N-glycosylated peptides were then eluted with 20%, 40% and 100% IPA in 5% acetic acid, respectively. Eluted peptides were then dried down and reconstituted in 0.1% formic acid for LC-MS/MS analysis.

Glycopeptides were analyzed using an Orbitrap Eclipse Tribrid mass spectrometer equipped with an RSLCnano system. A prepacked nano-LC column (15-cm length, 75-μm ID and filled with 3 μm C18 material) was used. LC-MS/MS runs were conducted for 180 mins. Precursor ion scan was acquired at 120,000 resolution in the Orbitrap analyzer, and precursors at a timeframe of 3 sec were selected for subsequent MS/MS fragmentation in the Orbitrap analyzer at 15,000 resolution. MS/MS fragmentation was conducted with stepped higher-energy collisional dissociation (HCD) product triggered collision induced dissociation (CID) (HCDpdCID). This method was set to trigger a CID experiment when oxonium ions indicative of glycosylation were seen in the HCD spectrum. Precursors with an unknown charge state, or charge state of +1 were excluded, and dynamic exclusion was enabled (30 sec duration). Glycopeptides were detected and assigned by Byonic software, Thermo Fisher Freestyle 1.8 and manual interpretation. Glycopeptides were searched against the FASTA sequence of the RBD region of the WT and Delta Spike proteins, and tryptic cleavage sites were searched (semispecific cleavage enabled). Oxidation of methionine, carbamidomethylation of cysteine and deamidation of asparagine and glutamine, as well as common human O-glycans were searched as variable modifications (Shajahan, A. et al., Glycobiology 2020 30.12: 981-988; Supekar, N. T. et al., Glycobiology 2021, 31 (9), 1080-1092; Natarajan, A. et al., Nat. Chem. Biol. 2020, 16 (10), 1062-1070).

### Data Analysis

To determine the fold change of lectin binding upon sample treatment (antibody or serum condition), ratio of untreated lectin control signals to treated conditions were calculated. Untreated controls were run as internal standards for each experiment. Hierarchical clustering heatmaps were generated using heatmaply package in RStudio (Galili, T. et al., Bioinformatics 2018, 34 (9), 1600-1602). Within the heatmaply package, row dendrograms were automatically calculated using the hclust function with a Euclidean distance measure and average linkage function.

### Glycan Array Data Analysis and Mapping

Publicly available glycan microarray data was obtained from Center for Functional Glycomics (CFG). Plant and fungal lectin microarray data was either processed at 1 μg/mL (AAL, Con A, DSL, GSL I, GSL II, Jacalin, LCA, LEL, PHA-E, PSA, RCA 120, SBA, SNA, STL, UEA I, VVL, WGA), 5 μg/mL (ECL), 10 μg/mL (DBA), or 100 μg/mL (LTL, MAL I, MAL II, PHA-L, PNA). Concentrations for analysis were determined to obtain comparable relative fluorescence units (RFU) signal above background from printed glycans on the mammalian glycan array v5.0 containing 611 glycans. FL-Spike RFUs were annotated from data in Watanabe et al. Minimum threshold for analysis was set at 5000 RFU. Force graphs and correlation mapping were projected using the GLAD online software.

### Results and Discussion

### A Lectin Toolkit to Map SARS-CoV-2 Spike Glycosylation

The Spike glycoprotein of SARS-CoV-2 contains overlapping yet distinct glycan compositions at each of its many glycosylation sites (Shajahan, A. et al., Glycobiology 2020 30.12: 981-988; Watanabe, Y. et al., Science 2020, eabb9983). Given advances in glycan profiling and diagnostics (Stowell, S. R. et al., Nat. Chem. Biol. 2014, 10 (6), 470-476; Wong, M. Y. et al., Proc. Natl. Acad. Sci. 2018, 115 (43), E10089-E10098; Zhang, L. et al., mAbs 2015, 8 (2), 205-215), studies were conducted to test whether non-antibody carbohydrate-binding proteins, or lectins, could detect changes in viral glycan microheterogeneity. Because glycans abound on the Spike glycoprotein and its RBD, studies were conducted to determine whether lectins would bind differentially and compete with neutralizing antibodies. Thus, experiments were performed to identify a collection of lectins to fingerprint the Spike RBD.

Studies were carried out using the Glycan Array Dashboard (GLAD) tool (Mehta, A. Y. and Cummings, R. D., Bioinforma. Oxf. Engl. 2019, 35 (18), 3536-3537) to collate the deposited glycan array datasets for 24 plant-and fungal-derived lectins against 611 glycans (Grant, O. C. et al., Glycobiology 2016, 26 (7), 772-783). Lectins displaying overlapping specificities and those containing distinct carbohydrate recognition properties were identified using correlation analysis (FIG. 2) and publicly available data (Bojar, D. et al., ACS Chem. Biol. 2022 17.11: 2993-3012). The glycan-binding specificities of each lectin were subsequently mapped onto the 46 unique glycoforms reported for each of the 74 binding sites on the trimeric ectodomain of recombinant full-length Spike (FL-Spike) to predict which lectins would recognize Spike glycans (FIG. 1D). From the glycan interaction mapping, it was expected that 14 lectins would bind with intermediate to strong affinities and 10 to interact minimally with FL-Spike.

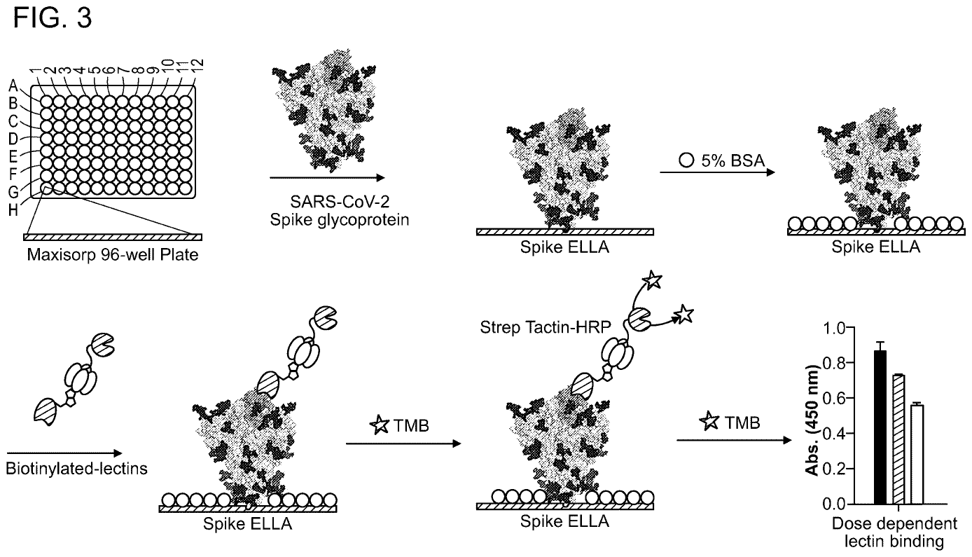

To test the in silico projections, an enzyme-linked lectin assay (ELLA) was optimized that would allow identification of which lectins from the curated suite bound FL-Spike and act as reporters of glycosylation. HEK-expressed FL-Spike was immobilized via passive adsorption to a hydrophilic 96-well plate. Biotinylated lectins were applied to the Spike protein-coated wells, and lectins bound to FL-Spike glycans were detected with StrepTactin-HRP conjugate (FIG. 3). FL-Spike has limited amounts of exposed galactose, N-acetylgalactosamine (GalNAc), and fucose (Fuc). Lectins recognizing these carbohydrates (Dolichos biflorus agglutinin (DBA, α-GalNAc), peanut agglutinin (PNA, Gal (β 1,3) GalNAc), (Ulex europaeus agglutinin I (UEAI, α-Fuc, arabinose), Griffonia simplicifolia lectin I (GSLI, α-Gal), Lotus tetragonolobus lectin (LTL, α-Fuc, arabinose)) showed minimal binding to FL-Spike.

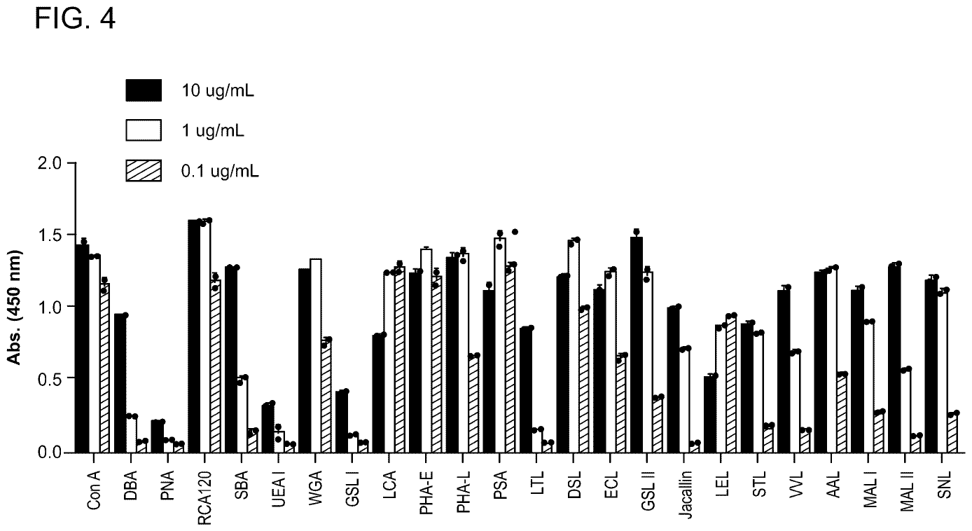

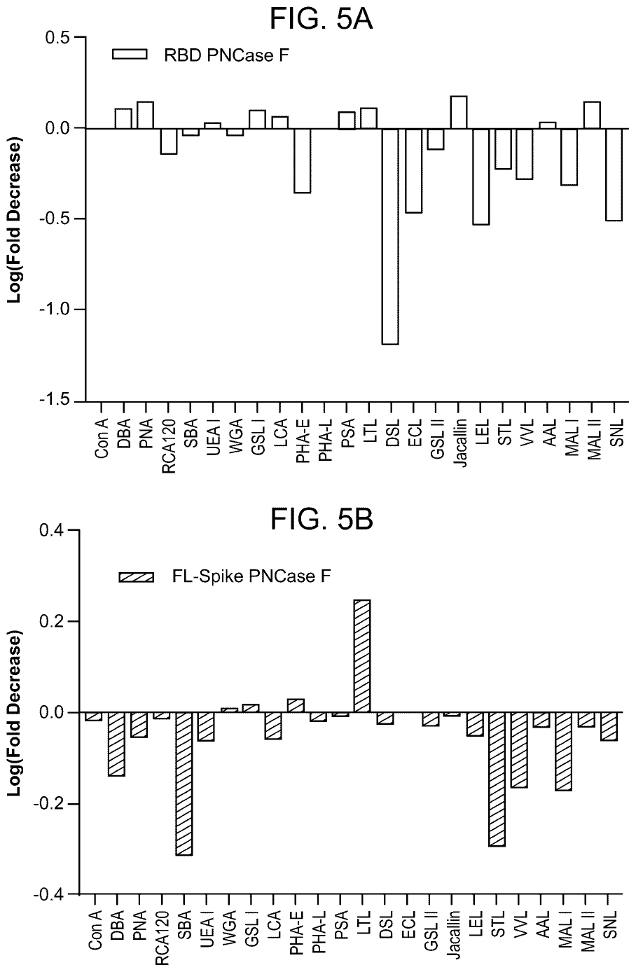

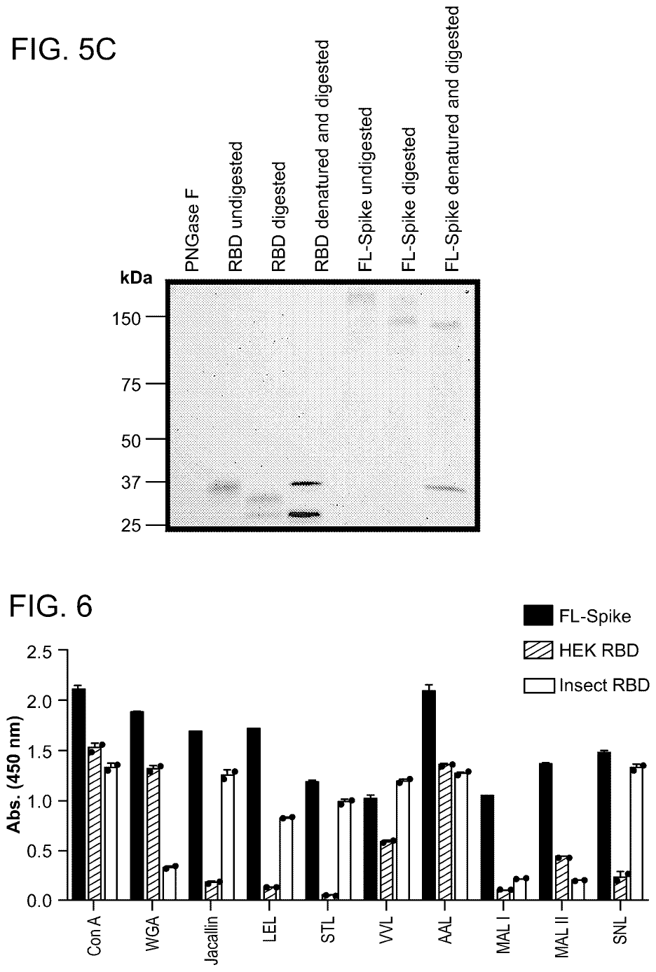

Of the 24 plant and fungal lectins tested, 19 bound FL-Spike with a moderate to strong signal. Lectin binding was dose-dependent (FIG. 4), indicating that complexation is specific. Additionally, treating FL-Spike with the N-glycosidase PNGase F reduced lectin binding, confirming that protein-glycan interactions drive the observed signal (FIG. 5). Several lectins predicted to bind weakly (Lycopersicon esculentum lectin (LEL, specificity for GlcNAc), Solanum tuberosum lectin (STL, GlcNAc), Vicia villosa lectin (VVL, GalNAc), Maackia amurensis lectin II (MALI, α-2,3-sialic acid), soybean agglutinin (SBA, Gal, GalNAc)) still gave moderate signals. The variance between predicted and experimental data may arise from changes in viral glycan display and minor differences in recombinant protein preparation. The results indicated that lectin valency also altered Spike recognition. For example, AAL, UEA I, and LTL could all interact with fucose residues, but AAL was the best Spike binder. AAL is a dimer with five fucose-binding sites per monomer (ten sites total). In contrast, UEA I is a dimer of monomers with one binding site each (two sites total), and LTL is a tetramer of monomers with one binding site each (four sites total). Thus, AAL, the lectin predicted to have the most multivalent interactions, exhibited the highest functional affinity.

### Lectin Fingerprints Differentially Report on SARS-CoV-2 Domains

Experiments were performed to evaluate the generality of lectin fingerprinting and its ability to report on glycosylation of different SARS-CoV-2 domains. It was posited that the receptor binding domain (RBD) of Spike would generate a lectin fingerprint distinct from the FL-Spike. Results indicated the RBD bound fewer lectins than FL-Spike (FIG. 1E). To determine how expression cell line affected glycosylation of Spike domains, the lectin fingerprint of recombinant RBD from insect cells was compared and distinct lectin binding signatures were identified, in agreement with recently published reports. (FIG. 65) (Zhang, Y. et al., Mol. Cell. Proteomics MCP 2020 20, 100058). These data indicated the lectin binding was specific and could rapidly report on differences in glycosylation for SARS-CoV-2 glycoproteins.

Neutralizing Antibodies Compete with Lectins for SARS-CoV-2 Spike

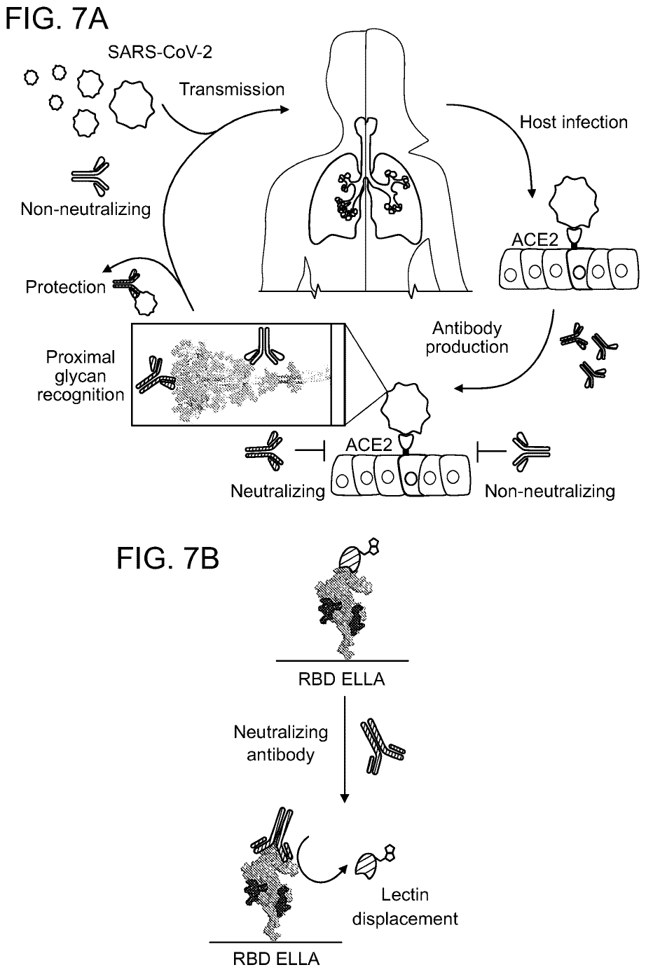

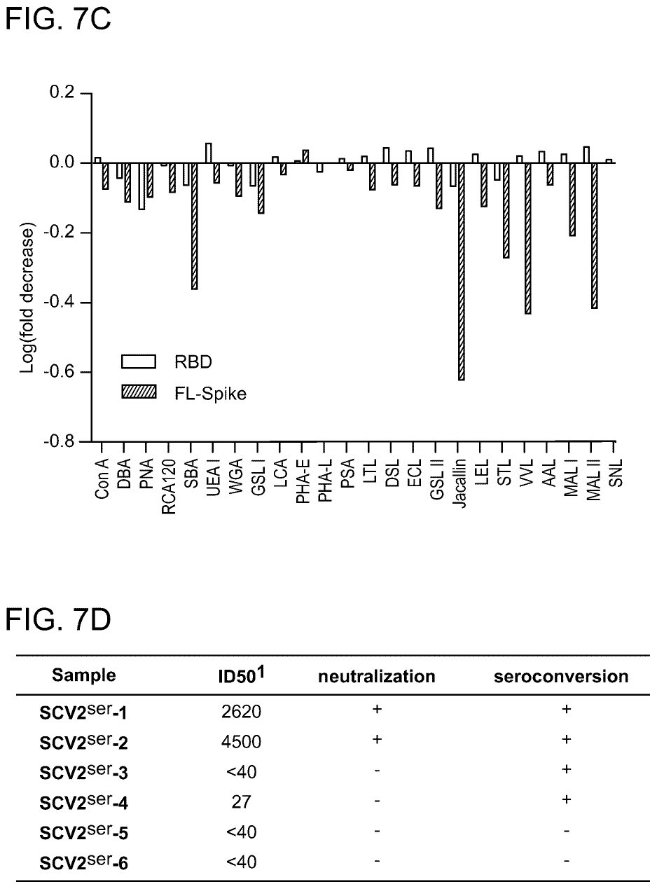

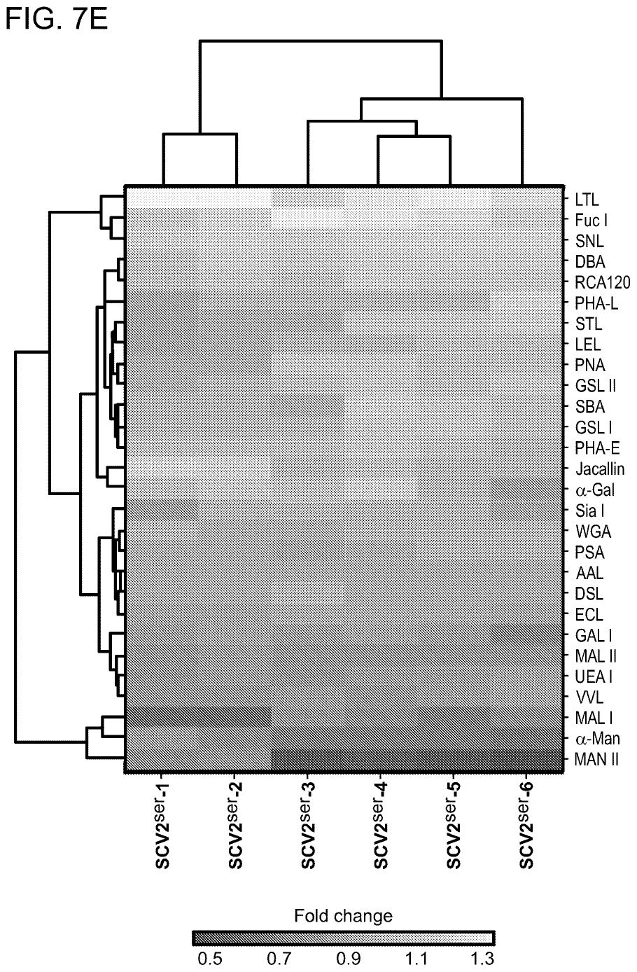

Upon infection with SARS-CoV-2, patients rapidly develop a humoral response to viral antigens (FIG. 7A). In many cases, the antibodies generated in response to SARS-CoV-2 infection are protective. Most characterized neutralizing antibodies engage the RBD, and many interact with the ACE2 orthosteric binding site (Zohar, T. and Alter, G., Nat. Rev. Immunol. 2020, 20 (7), 392-394). Proximal to the ACE2 binding site are two N-glycans, N331 and N343, and the corresponding glycans in the MERS-CoV and SARS-CoV-1 Spike protein contact neutralizing antibodies. Studies were performed to determine whether neutralizing antibodies would compete with lectins for Spike occupancy, leading to competitive displacement of lectins upon the addition of neutralizing antibody.

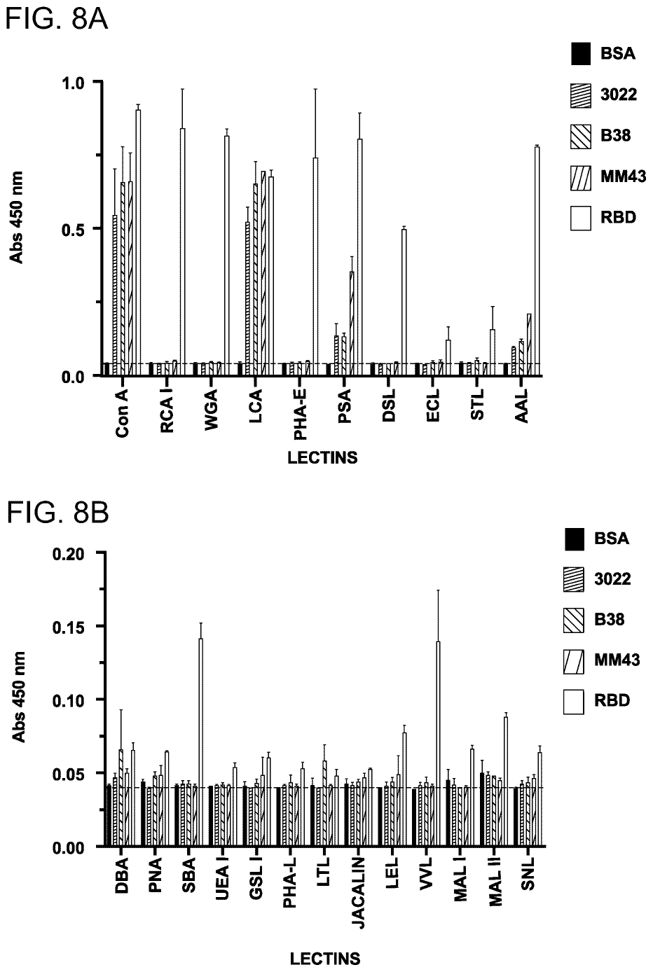

Antibodies themselves carry a single N-glycosylation mark that could complicate the interpretation of the lectin fingerprints (Jennewein, M. F. and Alter, G., Trends Immunol. 2017, 38 (5), 358-372). Initial studies were performed to examine lectin binding to immobilized commercial antibodies (1 μg/mL) and results indicated that, with the exception of ConA and LCA, minimal interactions were detected relative to those obtained using RBD binding (FIG. 8). These data indicated that antibody-RBD interactions, and not antibody glycans themselves, should be responsible for competitive fingerprints obtained upon antibody addition.

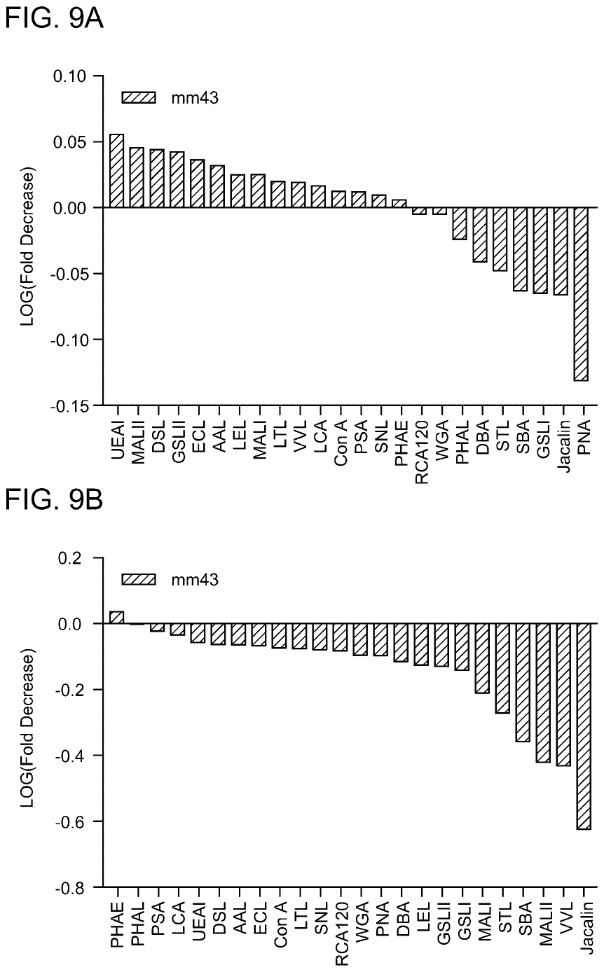

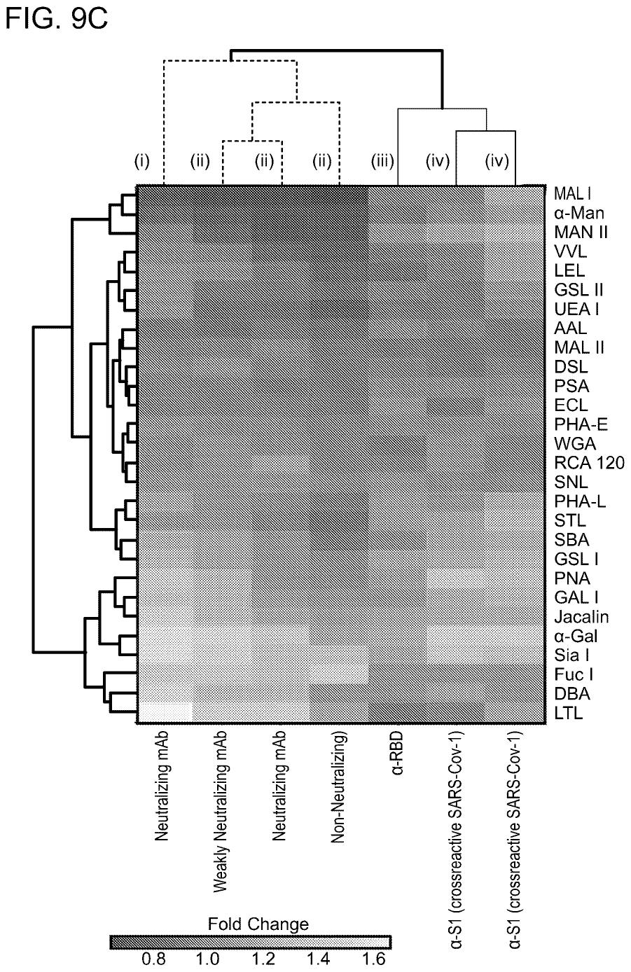

To test if antibodies compete with lectins that bind Spike glycans, an S1 neutralizing antibody raised against Val16-Arg685 of recombinant Spike (α-S1-MM43) was evaluated. The antibody's ability to displace lectins bound to immobilized RBD was assessed in a competitive ELLA assay (FIG. 7B). Competitive lectin fingerprints for the RBD differed when antibodies were present (FIG. 9A). A separate competitive fingerprint was generated for FL-Spike. Displacement patterns for the lectins jacalin (Gal(B1,3)GalNAc, O glycan), SBA (GalNAc, Gal) and STL (GlcNAc) were observed for both Spike and the RBD (FIG. 9B). A direct comparison (FIG. 7C) showed a greater decrease in lectin binding when antibodies were added to FL-Spike. Taken together, these findings indicate the assay is sensitive to the number of glycosites disrupted upon antibody binding (Henderson, R. et al., Nat. Struct. Mol. Biol. 2020, 27 (10), 925-933).

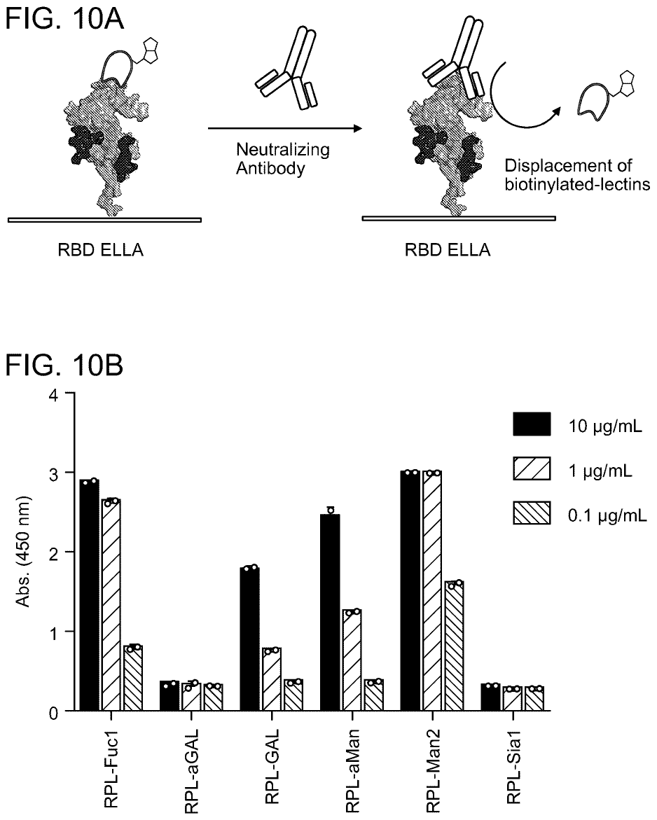

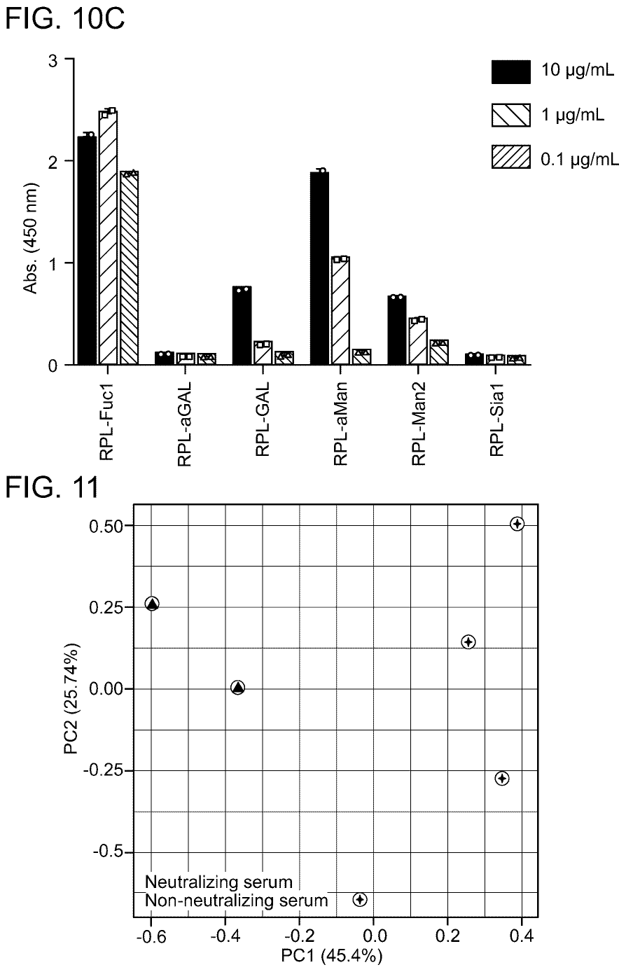

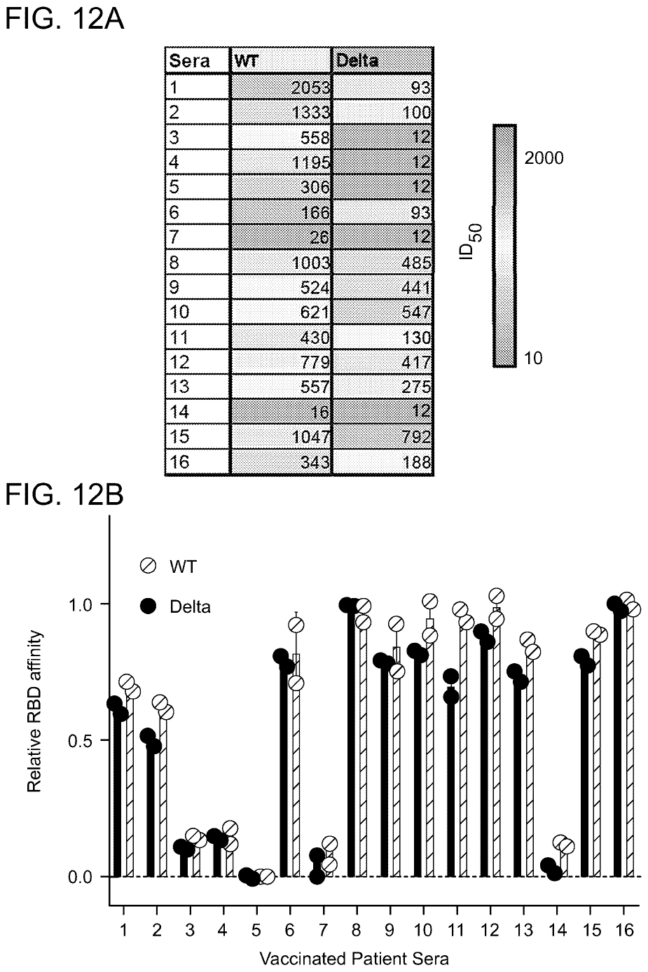

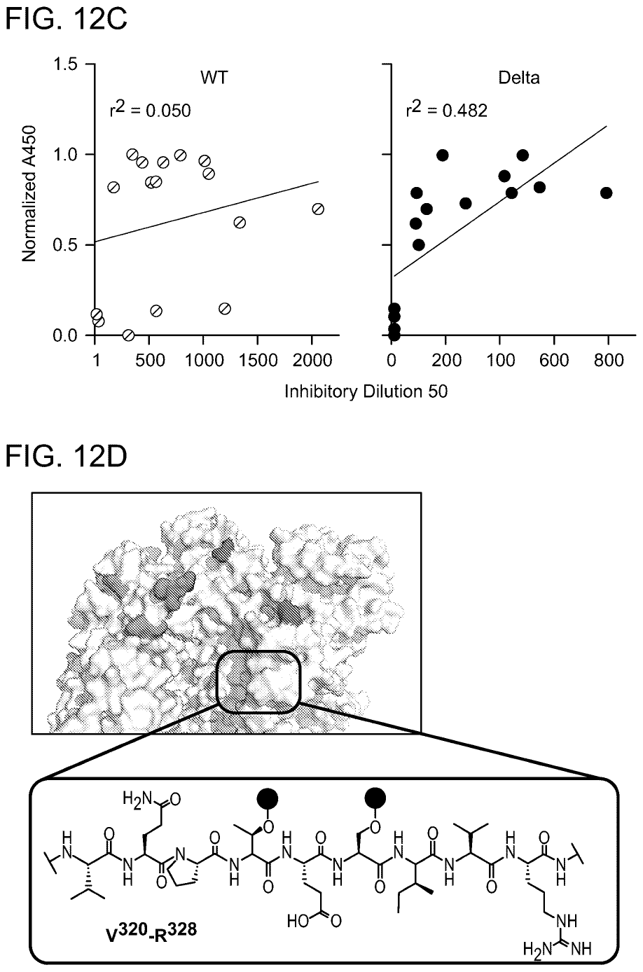

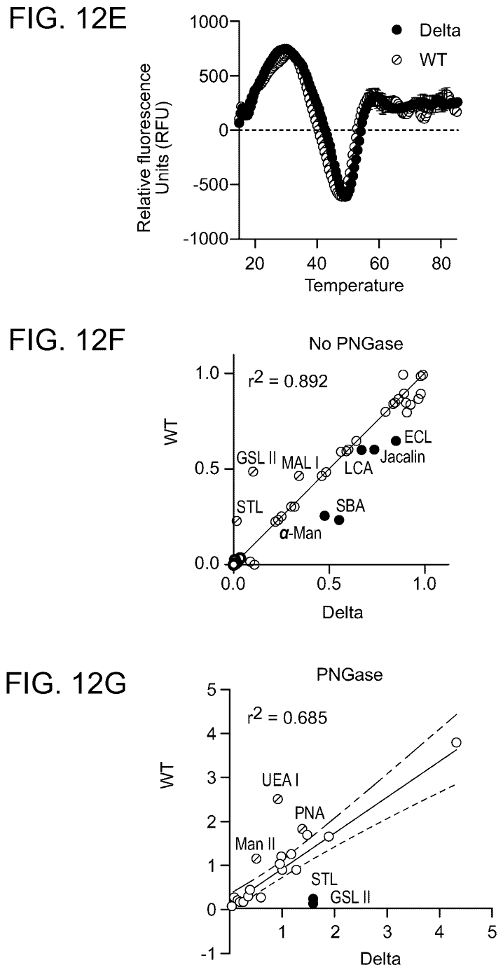

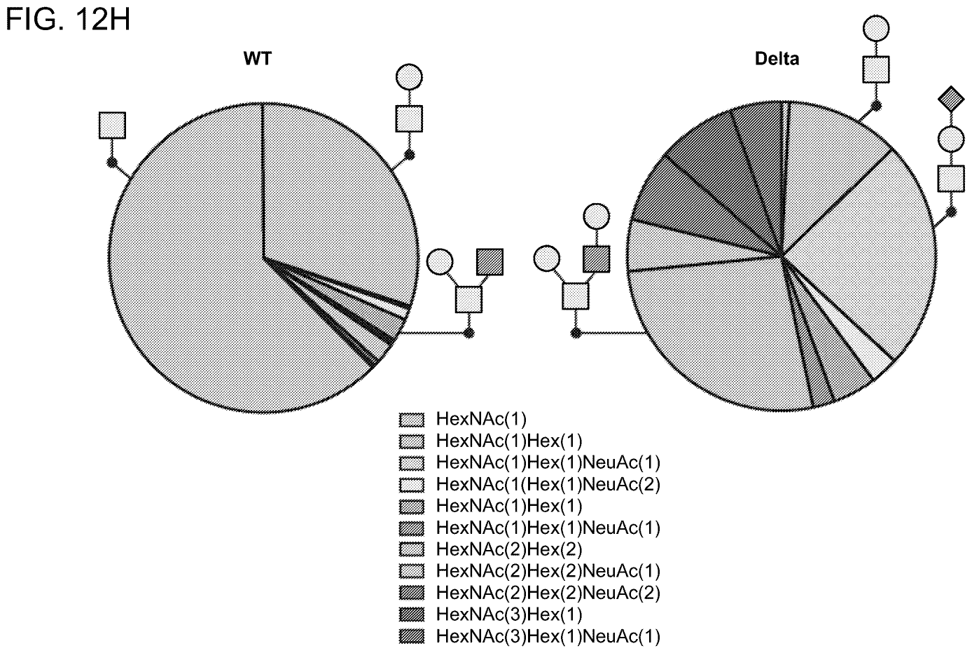

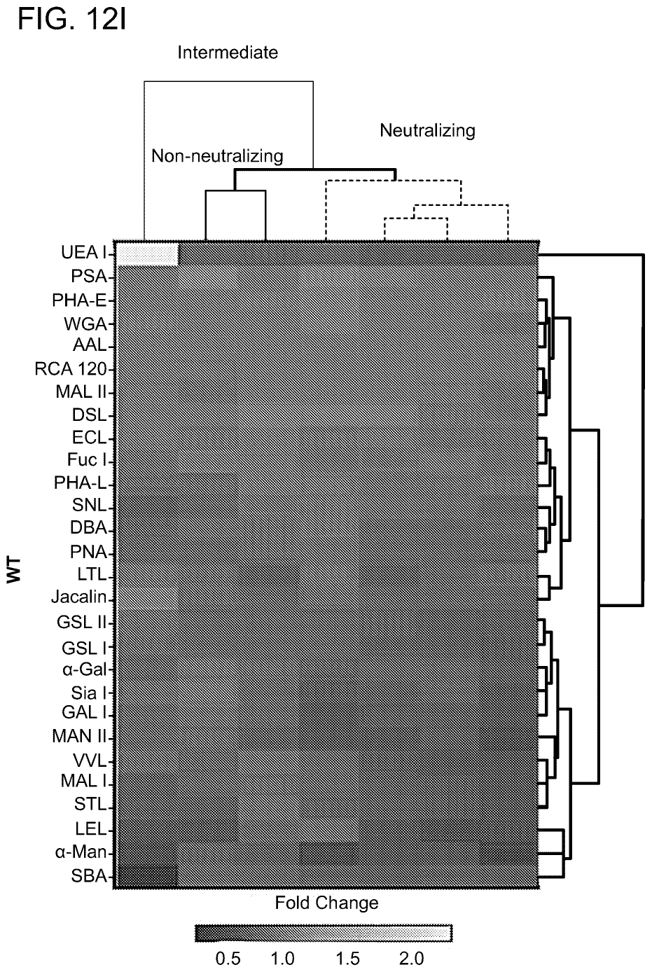

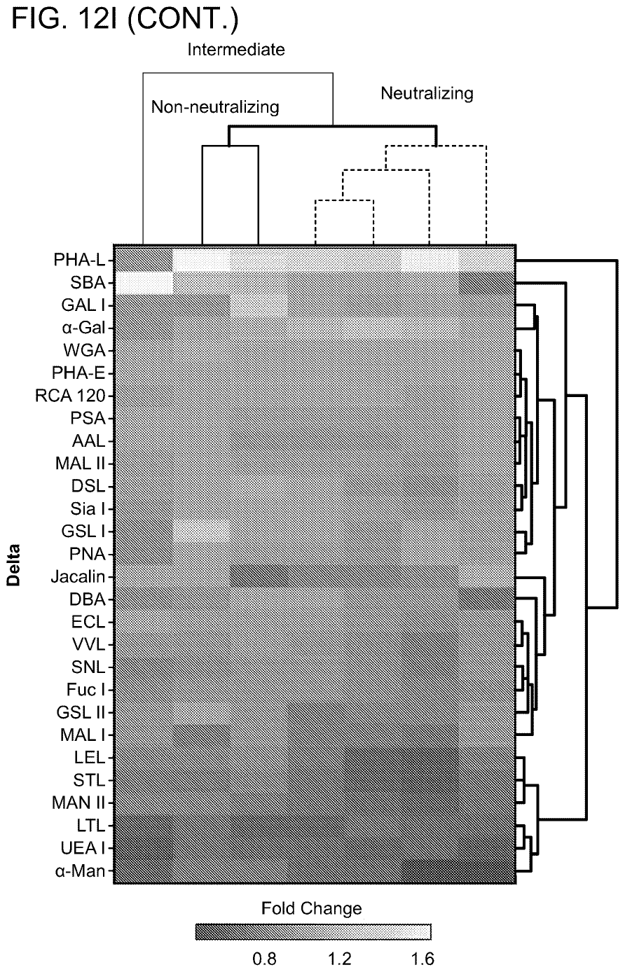

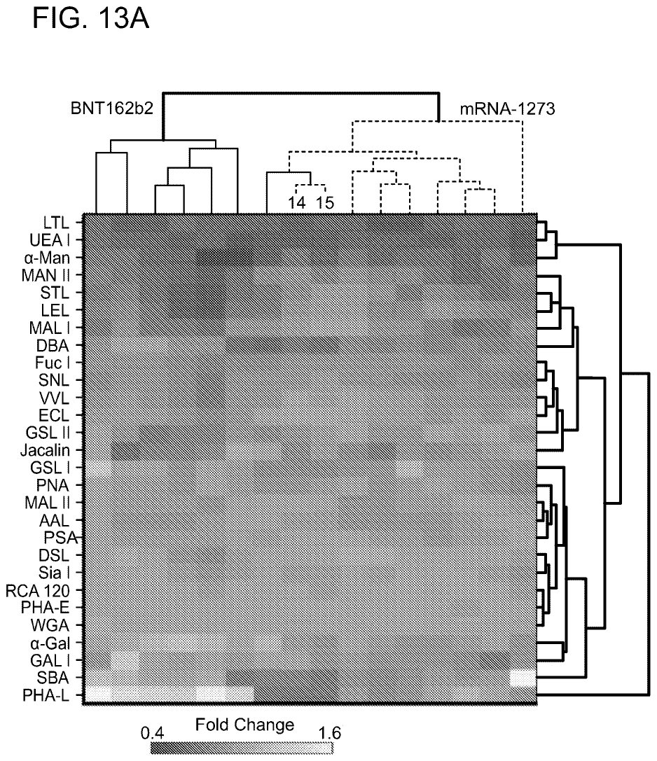

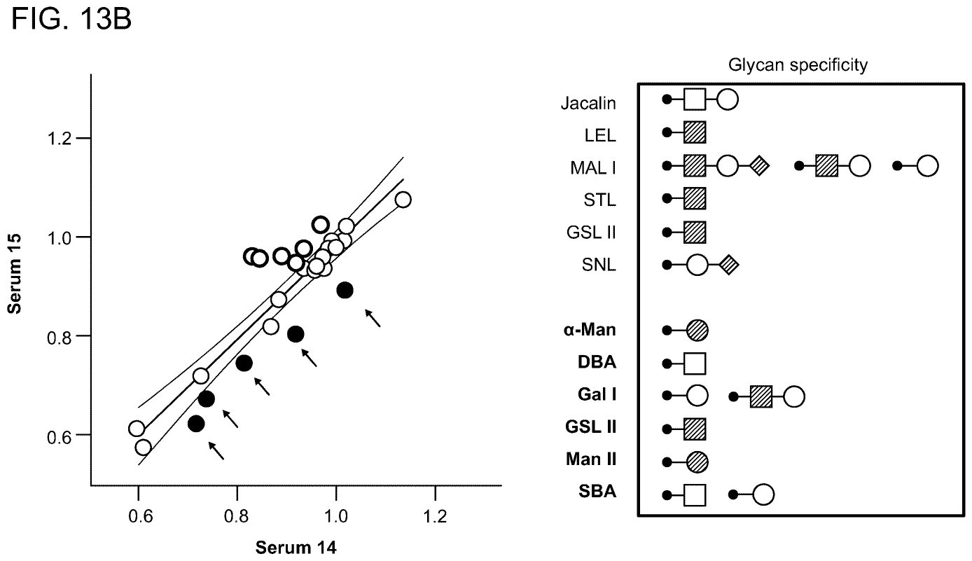

Studies were performed to determine whether a panel of lectins might distinguish neutralizing from non-neutralizing antibodies. Experiments queried whether neutralizing and non-neutralizing antibodies purified from convalescent patients would possess unique lectin fingerprints. In addition to the 24 plant and fungal lectins, six commercially available prokaryotic lectins with defined monosaccharide specificities (recombinant prokaryotic lectins, or RPLs, Table 1) were introduced to expand the detection of glycan microheterogeneity. The studies characterized the lectin's unique lectin fingerprints to FL-Spike and RBD (FIG. 10).

To avoid complications from FL-Spike structural and conformational plasticity, the RBD was used for competitive lectin fingerprints from patient antibodies. Four monoclonal antibodies purified from convalescent patients were tested (SCV2^{mAb}-1-SCV2^{mAb}-4) and two commercial mouse antibodies that were cross-reactive with SARS-CoV-1 RBD but were either known or likely to be non-neutralizing against SARS-C0v-2 (α-RBD-D002 and α-S1-CR3022). To reduce the high dimensionality of competitive lectin fingerprints, hierarchical clustering analysis with Euclidean distance measure (Galili, T. et al., Bioinformatics 2018, 34 (9), 1600-1602) was employed, where the similarity between individual lectin data was calculated combinatorially. The row and column dendrograms were automatically generated with seriation to find similarities in the lectin fingerprints (Hahsler, M. et al., J. Stat. Softw. 2008, 25, 1-34). Application of this protocol indicated that commercial neutralizing antibodies cluster separately from patient antibodies, indicating differences in lectin displacement. Within the patient antibody cluster, neutralizing antibody SCV2^{mAb}-1 grouped separately from the other patient antibodies. Weakly neutralizing antibody SCV2^{m-Ab}-2 and neutralizing antibody SCV2^{mAb}-3 clustered together, indicating a possible shared mechanism of binding that is distinct from SCV2^{mAb}-1. The non-neutralizing antibody, SCV2^{mAb}-4, clusters broadly with patient antibodies when compared with commercial antibodies, but separately from the other neutralizing patient antibodies (FIG. 9C). As indicated by the hierarchical clustering analysis, the profiles of α-S1 cross-reactive SARS-CoV-1 antibodies (α-RBD-D002 and α-S1-CR3022), which engaged RBD distal to the ACE2 binding site, were comparable (Yuan, M. et al., Science 2020, 368 (6491), 630-633). These results indicated that lectin fingerprinting could distinguish unique or overlapping binding modes of patient antibodies to Spike RBD.

### Convalescent Patient Sera Display Distinct Competitive Lectin Fingerprints

Rapid serological tests (Byrnes, J. R. et al., mSphere 2020, 5 (5)) to evaluate the neutralization capacity of patient antibodies would be valuable in evaluating antibody efficacy of vaccine formulations. It was reasoned that pre-vaccination patient-derived sera rather than purified antibodies could be used to generate unique lectin fingerprints predictive of antibody protectiveness. To this end, blinded serum was obtained from six convalescent patients. Two patients possessed neutralizing antibodies against WT SARS-CoV-2 (SCV2^{ser}-1, SCV2^{ser}-2), two had antibodies that bound WT Spike RBD but were non-neutralizing by an in vitro infection assay (SCV2^{ser}-3, SCV2^{ser}-4), and two generated antibodies that had no apparent Spike binding or neutralization activity (SCV2^{ser}-5, SCV2^{ser}-6) (FIG. 7D). Competitive lectin fingerprints were subjected to hierarchical clustering analysis by Euclidean measure (FIG. 7E) and by principal component analysis (PCA) (FIG. 11). In each analysis, the clustering patterns separated by sera type. Both samples with neutralizing serum clustered together, while non-neutralizing convalescent sera showed distinct lectin fingerprints. These initial findings indicated that competitive lectin fingerprinting could be benchmarked and used as a diagnostic tool for evaluating sera neutralizing potential.

### O-Glycosylation Differences in Spike Drive Antibody Efficacy in Vaccinated Patients

Robust neutralizing antibody responses against the W and other variants of concern (VOCs) have been generated from messenger RNA (mRNA)-based vaccination against SARS-CoV-2 Spike protein. Sera collected from individuals that received either one or two doses of mRNA-1273 and BNT162b2 mRNA vaccines (FIG. 12A, Table 2) was analyzed.

TABLE 2
Sera and analysis details

| | | | | | | | Days | | |
|---|---|---|---|---|---|---|---|---|---|
| | | | | | | | after | | |
| | Participant | | | | Vaccine | | last | A (wild | Delta/ |
| Sera | ID | Description | Age | Sex | manufacturer | Doses | dose | type) | B.1.617.2 |
| 1 | VIC-061 | VIC-061 - | 23 | F | Moderna | 2 | 59 | 2053 | 93 |
| | | Mod x2 | | | | | | | |
| | | (59 d) | | | | | | | |
| | | [23F] | | | | | | | |
| 2 | VIC-063 | VIC-063 - | 25 | M | Moderna | 2 | 60 | 1333 | 100 |
| | | Mod x2 | | | | | | | |
| | | (60 d) | | | | | | | |
| | | [25M] | | | | | | | |
| 3 | VIC-065 | VIC-065 - | 33 | M | Moderna | 2 | 117 | 558 | 12 |
| | | Mod x2 | | | | | | | |
| | | (117 d) | | | | | | | |
| | | [33M] | | | | | | | |
| 4 | VIC-069 | VIC-069 - | 28 | F | Pfizer | 2 | 2 | 1195 | 12 |
| | | Pfi x2 (2 d) | | | | | | | |
| | | [28F] | | | | | | | |
| 5 | VIC-070 | VIC-070 - | 58 | F | Moderna | 1 | 19 | 306 | 12 |
| | | Mod x1 | | | | | | | |
| | | (19 d) | | | | | | | |
| | | [58F] | | | | | | | |
| 6 | VIC-201 | VIC-201 - | 28 | F | Moderna | 1 | 21 | 166 | 93 |
| | | Mod x1 | | | | | | | |
| | | (21 d) | | | | | | | |
| | | [28F] | | | | | | | |
| 7 | VIC-203 | VIC-203 - | 46 | F | Moderna | 1 | 29 | 26 | 12 |
| | | Mod x1 | | | | | | | |
| | | (29 d) | | | | | | | |
| | | [46F] | | | | | | | |
| 8 | VIC-204 | VIC-204 - | 33 | F | Pfizer | 2 | 9 | 1003 | 485 |
| | | Pfi x2 (9 d) | | | | | | | |
| | | [33F] | | | | | | | |
| 9 | VIC-205 | VIC-205 - | 28 | F | Pfizer | 2 | 10 | 524 | 441 |
| | | Pfi x2 | | | | | | | |
| | | (10 d) | | | | | | | |
| | | [28F] | | | | | | | |
| 10 | VIC-206 | VIC-206 - | 37 | M | Pfizer | 2 | 9 | 621 | 547 |
| | | Pfi x2 (9 d) | | | | | | | |
| | | [37M] | | | | | | | |
| 11 | VIC-207 | VIC-207 - | 29 | F | Pfizer | 2 | 17 | 430 | 130 |
| | | Pfi x2 | | | | | | | |
| | | (17 d) | | | | | | | |
| | | [29F] | | | | | | | |
| 12 | VIC-208 | VIC-208 - | 29 | F | Pfizer | 2 | 17 | 779 | 417 |
| | | Pfi x2 | | | | | | | |
| | | (17 d) | | | | | | | |
| | | [29F] | | | | | | | |
| 13 | VIC-209 | VIC-209 - | 29 | M | Moderna | 2 | 108 | 557 | 275 |
| | | Mod x2 | | | | | | | |
| | | (108 d) | | | | | | | |
| | | [29M] | | | | | | | |
| 14 | VIC-210 | VIC-210 - | 46 | M | Moderna | 1 | 16 | 16 | 12 |
| | | Mod x1 | | | | | | | |
| | | (16 d) | | | | | | | |
| | | [46M] | | | | | | | |
| 15 | VIC-211 | VIC-211 - | 39 | F | Moderna | 1 | 27 | 1047 | 792 |
| | | Mod x1 | | | | | | | |
| | | (27 d) | | | | | | | |
| | | [39F] | | | | | | | |
| 16 | VIC-202 | VIC-202 - | 30 | F | Pfizer | 2 | 9 | 343 | 188 |
| | | Pfi x2 (9 d) | | | | | | | |
| | | [30F] | | | | | | | |

Collected sera elicited ID₅₀ values between 12 and 2053 in a SARS-CoV-2 infection assay with WT and Delta strains (FIG. 12A). The observed variability in neutralization was not reflected in sera affinity for the WT and Delta Spike RBD, which were similar across all samples by ELISA (FIG. 12B). No correlation (WT, r²=0.050; Delta, r²=0.482) was found between relative Spike RBD affinity and pseudovirus neutralization (FIG. 12C).

Viruses are notorious for altering their amino acid sequences during evolution. The effects of these changes on neighboring glycosylation sites are poorly understood. The Delta variant has two amino acid changes in its Spike RBD, L452R and T478K, (FIG. 12D) that are proximal to glycosites S323/T325 and N331 and N343. Experiments were performed to assess whether the changes in these residues could yield alterations in glycosylation that impact neutralization assays. The WT and Delta Spike variants had no differences in conformational stability as judged by differential scanning fluorimetry (FIG. 12E). Next, experiments were performed using lectin fingerprinting to examine changes in N- and O-glycosylation and results identified that WT and Delta glycosylation were highly correlated (r²=0.892).

Lectins with differences in their binding to WT versus Delta include ECL (specificity for Gal, GalNAc, Lac), GSL II (GlcNAc), Jacalin (sialylated T-antigen, Gal), LCA (Man, Glc), MAL I (Gal, Lac), SBA (Gal, GalNAc), STL (GlcNAc) and α-Man. These lectins recognize features of O-glycans suggesting WT and Delta strains have major differences in O-glycosylation (FIG. 12F). To confirm whether recognition was specific to 0-glycans, lectin fingerprinting was performed after removing N-glycans and results demonstrated enrichment in the binding of GSL II, STL, UEA I, PNA, and Man II (FIG. 12G). To determine if the lectin fingerprints were representative of direct O-glycan changes, comparative glycoproteomics were performed on WT and Delta Spike RBD (V320-R328). Although both variants contained GalNAc-Gal at this glycosite (WT, 30.2%; Delta, 12.0%), clear composition differences were obtained. The WT strain had mostly GalNAc at S323/T325 (62.3%) while Delta contained 24.1% GalNAc-Gal-NeuAc and 26.7% HexNAc(2)Hex(2), neither of which were observed in the WT variant. That SARS-CoV-2 alters its O-glycosylation to subvert host immunity is consistent with recent reports that the Omicron variant installs a novel O-glycosylation site to facilitate immune evasion. (S. Roberts, D. et al., Chem. Sci. 2022, 13 (36), 10944-10949)

Studies were performed to determine whether lectin fingerprinting could assess the neutralizing potential of vaccinated patient sera by analyzing samples from patients who received the BNT162b2 mRNA vaccine (FIG. 12I). Sera neutralization assays showed a broad distribution of neutralizing and non-neutralizing antibodies. For both WT and Delta RBD, it was found that neutralizing and non-neutralizing sera exhibited lectin fingerprints that were distinct from one another, but highly overlapping between samples within a given group. These data suggest that, within a cohort vaccinated with the same antigen, neutralization could be predicted by lectin fingerprinting against the WT and Delta.

Different mRNA Vaccines Yield Sera with Distinct Lectin Fingerprints

BNT162b2 and mRNA-1273 vaccination use different mRNA Spike sequences to elicit an antibody response (Kaplonek, P. et al., Sci. Transl. Med. 2022, eabm2311). To determine whether mRNA vaccination strategy changes how vaccinated patient sera engage Spike RBD, lectin fingerprinting was performed on all patient sera (n=16). Hierarchical clustering analysis of lectin fingerprinting showed dissimilar patterns from the BNT162b2 and mRNA-1273 vaccinees, suggesting differential vaccination strategies elicit variations in Spike RBD glycoforms. The results demonstrated lectin fingerprints that varied with vaccination strategy (FIG. 13A). Patients 14 and 15 received the mRNA-1273 vaccine, and serum from 15 could potently neutralize all VOCs tested whereas that from patient 14 did not neutralize any VOC (Table 1). Additional studies were conducted that included a lectin fingerprint correlation analysis (FIG. 13B). Lectin fingerprinting revealed glycan binding for serum 15 was enriched for lectins that engage GlcNAc and N-acetylneuraminic acid, whereas serum 14 enriched for galactose- and mannose-binding lectins. These data suggest that the broadly neutralizing serum 15 engages Spike RBD epitopes proximal to 0-glycans while broadly non-neutralizing serum 14 engages protein epitopes between N- and O-glycans.

### DISCUSSION

As SARS-CoV-2 variants emerge and evolve for immune escape, changes in glycosite composition as well as the appearance of new glycosites (Nabel, K. G.; et al., Science 2022, 375 (6578), eabl6251) will inevitably impact antibody recognition. Few low-cost tools exist to rapidly profile protein glycosylation and report on glycoprotein-protein interactions (gPPIs). In the case of SARS-CoV-2 infections, where glycosylation of the Spike protein plays a central role in viral infection and immune evasion, such tools would illuminate how viral protein glycosylation changes over the course of infection and its inevitable evolution between hosts. In this study, we developed a lectin-fingerprinting method that provides a snapshot of glycan microheterogeneity on recombinant FL-Spike and its RBD.

To generate this lectin fingerprint, 24 plant and fungal lectins and six prokaryotic lectins were used to index the accessible glycans on the viral protein surfaces. Lectin fingerprints for FL-Spike and RBD were consistent with mass spectrometric analysis of SARS-CoV-2 glycoproteins, demonstrating the accuracy of lectin fingerprinting as a readout of protein glycosylation. Additionally, lectin fingerprinting rapidly reported on differences in glycosylation for SARS-CoV-2 glycoproteins produced in different cell types. Together the data indicated this method is useful for evaluating how alterations to protein glycosylation during proteostatic stress (e.g. influenza infection), or in viral infections with multi-tissue tropism that could contribute to viral glycan heterogeneity, depending on the cell type propagating the virus (Wong, M. Y. et al., Proc. Natl. Acad. Sci. 2018, 115 (43), E10089-E10098). Unlike other methods for profiling protein glycosylation (e.g., MS analysis, lectin microarrays), lectin fingerprinting requires minimal sample processing and uses common laboratory equipment and reagents. Furthermore, this method can be readily scaled up for high-throughput screening applications and easily formatted for probing any glycoprotein of interest.

Recent reports indicate that the majority of anti-SARS-CoV-2 neutralizing antibodies target the RBD. Still, not all antibodies generated against RBD are neutralizing. This suggests that privileged epitopes exist on RBD and targeting of these sites by a host may confer increased immunity against the virus. The assays currently used to examine binding of convalescent patient antibodies to RBD cannot distinguish which sites on RBD are bound, nor which antibodies are neutralizing. Yet structural studies on antibody-Spike interactions suggest that antibodies make contacts with glycans on RBD. This indicates that tools capable of distinguishing antibody engagement of RBD could offer a new route to evaluate convalescent patient antibodies and their likelihood of conferring protection from SARS-CoV-2 infection. Such reagents could also uncover mechanisms of glycan-mediated immune evasion.

If anti-SARS-CoV-2 antibodies engage epitopes on Spike RBD proximal to its glycosites, antibodies should be able to competitively displace bound lectins and perturb the lectin-fingerprint. Indeed, results of studies described herein indicated that commercially available neutralizing antibodies bound proximally to exposed glycosites and perturbed the lectin fingerprints. Upon binding RBD, non-neutralizing antibodies also perturbed the lectin fingerprint, but in a manner that was distinct from neutralizing antibodies. These data indicate that, in engaging their respective epitopes, antibodies competitively displace lectins in a pattern that corresponds to their neutralization capacity. Lectin fingerprinting was employed to evaluate the neutralization potential of antibodies within pre-vaccination convalescent patient sera. The use of patient sera is more relevant for clinical evaluations but demands the evaluation of antibodies within a more complex mixture of serum glycoproteins. Studies described herein demonstrated that convalescent sera yielded distinct lectin fingerprints that corresponded to their neutralization efficacy.

Glycan changes have been implicated in stabilizing Spike protein structure and influencing immune selective pressure (Harbison, A. M. et al., Chem. Sci. 13 (2), 386-395; Kang, L. et al., Cell 2021, 184 (17), 4392-4400.e4). Differences in S323/T325 O-glycosylation in the Spike RBD of WT and Delta variants highlights a potentially critical nexus in the evasion of SARS-CoV-2. When O-glycosylation was reduced, as seen in the mutation of T372A from scanned SARS-CoV-2 genomes, infectious virus showed increase replication (Kang, L. et al., Cell 2021, 184 (17), 4392-4400.e4) suggesting a delicate balance of glycosylation status is critical for maintaining immune evasion and infectivity. The differences in antibody responses elicited from BNT162b2 and mRNA-1273 that encode different Spike antigens result in changes in glycosylation. How population heterogeneity selects for specific glycan microheterogeneity may influence the evolution of SARS-CoV-2.

Data and results from studies described herein indicated that lectin fingerprinting can be used as a diagnostic tool for evaluating antibody efficacy against SARS-CoV-2 antigens and other glycosylated proteins. It is believed methods described herein can be used to profile alterations in SARS-CoV-2 glycosylation that are present in rapidly emerging, highly infectious VOCs. Understanding how changes in glycan structure influence antibody recognition may reveal new insights into how new viral variants are able to evolve to evade immune responses. More broadly, results of studies described herein support a conclusion that lectin fingerprinting can be used to map protein glycosylation for other proteins in which glycosylation plays a key functional role.

### EQUIVALENTS

Although several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

All references, patents and patent applications and publications that are cited or referred to in this application are incorporated by reference in their entirety herein.

What is claimed is:

1. A method of identifying a virus-neutralization status of a candidate antiviral agent, the method comprising:
(a) contacting a virion of a virus that has previously contracted an anti-virus antibody, with one or more preselected glycan-binding agents lectin molecules, wherein the contacting results in a virion/glycan-binding agent virion/lectin molecule complex;
(b) contacting the virion/glycan-binding agent virion/lectin molecule complex with a candidate antiviral agent that is a candidate to block infection with the virus; and
(c) determining one or more binding characteristics of the preselected glycan-binding agent(s) lectin molecule(s) with the antibody-contacted virion subsequent to the contacting with the candidate antiviral agent, wherein the determined one or more binding characteristics identifies the virus-neutralization status of the antiviral agent.

2. The method of claim 57 wherein the one or more binding characteristics are determined for binding of the one or more preselected lectin molecules with a virion protein.

3. The method of claim 1, wherein the candidate agent to block infection with the virus is an anti-virus antibody.

4. (canceled)

5. The method of claim 1, wherein neutralizing the virus comprises blocking infection of the subject by the virus, optionally wherein neutralizing the virus comprises blocking entry of the viral particle of the virus into a cell.

6. (canceled)

7. The method of claim 57 wherein the determined binding characteristics comprise one or more of (a) the presence or absence of binding of the one or more preselected lectin molecule(s) with the virion protein; (b) a level of binding of the one or more preselected lectin molecules with the virion protein; (c) one or more location of the binding of the one or more preselected lectin molecules on the virion protein; (d) identity of the one or more preselected lectin molecules that bind the virion protein; and (e) identity of the one or more preselected lectin molecules that do not bind the virion protein.

8. The method of claim 1, further comprising comparing binding characteristics determined in step (c) with control binding characteristics to identify the virus-neutralization status of the antiviral agent.

9. The method of claim 8, wherein the control binding characteristics comprise the binding characteristics of the lectins in the virion/lectin molecule complex prior to contact with the candidate antiviral agent.

10. The method of claim 1, wherein the anti-viral antibody is obtained from a first subject known to have had been infected by the virus, optionally wherein the anti-viral antibody is obtained from convalescent serum obtained from the first subject.

11. (canceled)

12. The method of claim 1, wherein the anti-viral antibody is obtained from a first subject known to not have been infected by the virus but to have received one or more vaccinations against the virus.

13. The method of claim 1, wherein the anti-virus antibody is a manufactured antibody, optionally wherein the anti-virus antibody is a recombinant antibody.

14-18. (canceled)

19. The method of claim 1, wherein the virus is an RNA virus.

20-23. (canceled)

24. The method of claim 1, wherein the virus is an enveloped virus.

25. The method of claim 57, wherein the one or more preselected lectin molecules comprise one or more eukaryotic lectins, prokaryotic lectins, and plant lectins, optionally, wherein the one or more preselected lectin molecules comprise one or more mammalian lectins.

26. (canceled)

27. The method of claim 1, wherein a method of determining the one or more binding characteristics comprises an enzyme-linked lectin assay (ELLA).

28. (canceled)

29. The method of claim 57 wherein one or more of the preselected lectin molecules is detectably labeled.

30-32. (canceled)

33. The method of claim 57, wherein the virus is a SARS-CoV-2 virus, the anti-virus antibody is a purified antibody, and the one or more preselected lectin molecules contacting the virion protein comprise: MALI, RPL-αMan, RPL-Man2, PNA, RPL-Gal1, Jacalin, RPL-αGal, RPL-Sia1, RPL-Fuc1, DBA, and LTL, and wherein determining the presence of binding of the one or more preselected lectin molecules MALI, RPL-αMan, RPL-Man2, PNA, RPL-Gal1, Jacalin, RPL-αGal, RPL-Sia1, RPL-Fuc1, DBA, and LTL to the virion protein identifies the anti-virus antibody as a SARS-CoV-2-neutralizing antibody, and optionally further comprising administering the anti-viral antibody identified as neutralizing the virus to a subject as a treatment for a SARS-CoV-2 viral infection in the subject.

34. (canceled)

35. The method of claim 57, wherein the virus is a SARS-CoV-2 virus, the anti-viral antibody is obtained from a convalescent first subject patient serum sample, and the one or more preselected lectin molecules contacting the virion comprise: ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1, and wherein determining the presence of binding of the one or more preselected lectin molecules ConA, DBA, RCA I, PNA, SBA, UEA I, WGA, GSL I, LCA, PHA-E, PHA-L, PSA, LTL, DSL, ECL, GSL II, Jacalin, LEL, STL, VVL, AAL, MAL I, MAL II, SNL, RPL-αGal, RPL-Gal1, RPL-αMan, RPL-Man2, RPL-Sia1, and RPL-Fuc1 to the virion protein identifies the anti-virus antibody as a SARS-CoV-2-neutralizing antibody, optionally, further comprising administering the anti-viral antibody identified as neutralizing the virus to a second subject as a treatment for a SARS-CoV-2 viral infection in the second subject.

36-38. (canceled)

39. A method of identifying a candidate antibody to treat an infection by a SARS-CoV-2 virus, the method comprising:
(a) determining whether an anti-virus antibody has a status of neutralizing the virus using a method set forth in claim 1;
(b) further assessing the anti-virus antibody determined to have the status of neutralizing the virus for efficacy and safety; wherein the further assessment comprises:
(i) contacting a plurality of cells infected with the SARS-CoV-2 virus with the anti-viral antibody determined to have the neutralizing status;
(ii) administering the anti-viral antibody determined to have the neutralizing status to one or a plurality of subjects known to be infected with the SARS-CoV-2 virus;
(c) assessing the SARS-CoV-2 virus infection in the cell or subject following the contacting and administering, respectively; and
(d) determining from the assessment a presence and/or level of efficacy of the SARS-CoV-2 anti-virus antibody treatment of the SARS-CoV-2 infection in the cell or subject, respectively, wherein one or both of the presence and efficacy identifies the SARS-CoV-2 anti-virus antibody as a candidate antibody to treat an infection by the SARS-CoV-2 virus.

40. An enzyme-linked lectin assay (ELLA), comprising:
a plurality of preselected lectins that bind a virus protein.

41-56. (canceled)

57. The method of claim 1, wherein the preselected glycan-binding agent is a preselected glycan-binding protein, optionally is a preselected glycan-binding antibody, a preselected glycan-binding antibody fragment, a preselected lectin, or a preselected glycan-binding nanobody that binds a specific glycan epitope.


SEQUENCE LISTING (8 sequences)

SEQ ID NO: 1 - protein, 223 aa, synthetic construct
MRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKGAGSSLEVLFQ

SEQ ID NO: 2 - protein, 7096 aa, Coronavirus
  source 1..7096: note = Cornoavirus 2 isolate 2019-nCoV_HKU-SZ-005b_2020
MESLVPGFNEKTHVQLSLPVLQVRDVLVRGFGDSVEEVLSEARQHLKDGTCGLVEVEKGVLPQLEQPYVFIKRSDARTAPHGHVMVELVAELEGIQYGRSGETLGVLVPHVGEIPVAYRKVLLRKNGNKGAGGHSYGADLKSFDLGDELGTDPYEDFQENWNTKHSSGVTRELMRELNGGAYTRYVDNNFCGPDGYPLECIKDLLARAGKASCTLSEQLDFIDTKRGVYCCREHEHEIAWYTERSEKSYELQTPFEIKLAKKFDTFNGECPNFVFPLNSIIKTIQPRVEKKKLDGFMGRIRSVYPVASPNECNQMCLSTLMKCDHCGETSWQTGDFVKATCEFCGTENLTKEGATTCGYLPQNAVVKIYCPACHNSEVGPEHSLAEYHNESGLKTILRKGGRTIAFGGCVFSYVGCHNKCAYWVPRASANIGCNHTGVVGEGSEGLNDNLLEILQKEKVNINIVGDFKLNEEIAIILASFSASTSAFVETVKGLDYKAFKQIVESCGNFKVTKGKAKKGAWNIGEQKSILSPLYAFASEAARVVRSIFSRTLETAQNSVRVLQKAAITILDGISQYSLRLIDAMMFTSDLATNNLVVMAYITGGVVQLTSQWLTNIFGTVYEKLKPVLDWLEEKFKEGVEFLRDGWEIVKFISTCACEIVGGQIVTCAKEIKESVQTFFKLVNKFLALCADSIIIGGAKLKALNLGETFVTHSKGLYRKCVKSREETGLLMPLKAPKEIIFLEGETLPTEVLTEEVVLKTGDLQPLEQPTSEAVEAPLVGTPVCINGLMLLEIKDTEKYCALAPNMMVTNNTFTLKGGAPTKVTFGDDTVIEVQGYKSVNITFELDERIDKVLNEKCSAYTVELGTEVNEFACVVADAVIKTLQPVSELLTPLGIDLDEWSMATYYLFDESGEFKLASHMYCSFYPPDEDEEEGDCEEEEFEPSTQYEYGTEDDYQGKPLEFGATSAALQPEEEQEEDWLDDDSQQTVGQQDGSEDNQTTTIQTIVEVQPQLEMELTPVVQTIEVNSFSGYLKLTDNVYIKNADIVEEAKKVKPTVVVNAANVYLKHGGGVAGALNKATNNAMQVESDDYIATNGPLKVGGSCVLSGHNLAKHCLHVVGPNVNKGEDIQLLKSAYENFNQHEVLLAPLLSAGIFGADPIHSLRVCVDTVRTNVYLAVFDKNLYDKLVSSFLEMKSEKQVEQKIAEIPKEEVKPFITESKPSVEQRKQDDKKIKACVEEVTTTLEETKFLTENLLLYIDINGNLHPDSATLVSDIDITFLKKDAPYIVGDVVQEGVLTAVVIPTKKAGGTTEMLAKALRKVPTDNYITTYPGQGLNGYTVEEAKTVLKKCKSAFYILPSIISNEKQEILGTVSWNLREMLAHAEETRKLMPVCVETKAIVSTIQRKYKGIKIQEGVVDYGARFYFYTSKTTVASLINTLNDLNETLVTMPLGYVTHGLNLEEAARYMRSLKVPATVSVSSPDAVTAYNGYLTSSSKTPEEHFIETISLAGSYKDWSYSGQSTQLGIEFLKRGDKSVYYTSNPTTFHLDGEVITFDNLKTLLSLREVRTIKVFTTVDNINLHTQVVDMSMTYGQQFGPTYLDGADVTKIKPHNSHEGKTFYVLPNDDTLRVEAFEYYHTTDPSFLGRYMSALNHTKKWKYPQVNGLTSIKWADNNCYLATALLTLQQIELKFNPPALQDAYYRARAGEAANFCALILAYCNKTVGELGDVRETMSYLFQHANLDSCKRVLNVVCKTCGQQQTTLKGVEAVMYMGTLSYEQFKKGVQIPCTCGKQATKYLVQQESPFVMMSAPPAQYELKHGTFTCASEYTGNYQCGHYKHITSKETLYCIDGALLTKSSEYKGPITDVFYKENSYTTTIKPVTYKLDGVVCTEIDPKLDNYYKKDNSYFTEQPIDLVPNQPYPNASFDNFKFVCDNIKFADDLNQLTGYKKPASRELKVTFFPDLNGDVVAIDYKHYTPSFKKGAKLLHKPIVWHVNNATNKATYKPNTWCIRCLWSTKPVETSNSFDVLKSEDAQGMDNLACEDLKPVSEEVVENPTIQKDVLECNVKTTEVVGDIILKPANNSLKITEEVGHTDLMAAYVDNSSLTIKKPNELSRVLGLKTLATHGLAAVNSVPWDTIANYAKPFLNKVVSTTTNIVTRCLNRVCTNYMPYFFTLLLQLCTFTRSTNSRIKASMPTTIAKNTVKSVGKFCLEASFNYLKSPNFSKLINIIIWFLLLSVCLGSLIYSTAALGVLMSNLGMPSYCTGYREGYLNSTNVTIATYCTGSIPCSVCLSGLDSLDTYPSLETIQITISSFKWDLTAFGLVAEWFLAYILFTRFFYVLGLAAIMQLFFSYFAVHFISNSWLMWLIINLVQMAPISAMVRMYIFFASFYYVWKSYVHVVDGCNSSTCMMCYKRNRATRVECTTIVNGVRRSFYVYANGGKGFCKLHNWNCVNCDTFCAGSTFISDEVARDLSLQFKRPINPTDQSSYIVDSVTVKNGSIHLYFDKAGQKTYERHSLSHFVNLDNLRANNTKGSLPINVIVFDGKSKCEESSAKSASVYYSQLMCQPILLLDQALVSDVGDSAEVAVKMFDAYVNTFSSTFNVPMEKLKTLVATAEAELAKNVSLDNVLSTFISAARQGFVDSDVETKDVVECLKLSHQSDIEVTGDSCNNYMLTYNKVENMTPRDLGACIDCSARHINAQVAKSHNIALIWNVKDFMSLSEQLRKQIRSAAKKNNLPFKLTCATTRQVVNVVTTKIALKGGKIVNNWLKQLIKVTLVFLFVAAIFYLITPVHVMSKHTDFSSEIIGYKAIDGGVTRDIASTDTCFANKHADFDTWFSQRGGSYTNDKACPLIAAVITREVGFVVPGLPGTILRTTNGDFLHFLPRVFSAVGNICYTPSKLIEYTDFATSACVLAAECTIFKDASGKPVPYCYDTNVLEGSVAYESLRPDTRYVLMDGSIIQFPNTYLEGSVRVVTTFDSEYCRHGTCERSEAGVCVSTSGRWVLNNDYYRSLPGVFCGVDAVNLLTNMFTPLIQPIGALDISASIVAGGIVAIVVTCLAYYFMRFRRAFGEYSHVVAFNTLLFLMSFTVLCLTPVYLFLPGVYSVIYLYLTFYLTNDVSFLAHIQWMVMFTPLVPFWITIAYIICISTKHFYWFFSNYLKRRVVFNGVSFSTFEEAALCTFLLNKEMYLKLRSDVLLPLTQYNRYLALYNKYKYFSGAMDTTSYREAACCHLAKALNDFSNSGSDVLYQPPQTSITSAVLQSGFRKMAFPSGKVEGCMVQVTCGTTTLNGLWLDDVVYCPRHVICTSEDMLNPNYEDLLIRKSNHNFLVQAGNVQLRVIGHSMQNCVLKLKVDTANPKTPKYKFVRIQPGQTFSVLACYNGSPSGVYQCAMRPNFTIKGSFLNGSCGSVGFNIDYDCVSFCYMHHMELPTGVHAGTDLEGNFYGPFVDRQTAQAAGTDTTITVNVLAWLYAAVINGDRWFLNRFTTTLNDFNLVAMKYNYEPLTQDHVDILGPLSAQTGIAVLDMCASLKELLQNGMNGRTILGSALLEDEFTPFDVVRQCSGVTFQSAVKRTIKGTHHWLLLTILTSLLVLVQSTQWSLFFFLYENAFLPFAMGIIAMSAFAMMFVKHKHAFLCLFLLPSLATVAYFNMVYMPASWVMRIMTWLDMVDTSLSGFKLKDCVMYASAVVLLILMTARTVYDDGARRVWTLMNVLTLVYKVYYGNALDQAISMWALIISVTSNYSGVVTTVMFLARGIVFMCVEYCPIFFITGNTLQCIMLVYCFLGYFCTCYFGLFCLLNRYFRLTLGVYDYLVSTQEFRYMNSQGLLPPKNSIDAFKLNIKLLGVGGKPCIKVATVQSKMSDVKCTSVVLLSVLQQLRVESSSKLWAQCVQLHNDILLAKDTTEAFEKMVSLLSVLLSMQGAVDINKLCEEMLDNRATLQAIASEFSSLPSYAAFATAQEAYEQAVANGDSEVVLKKLKKSLNVAKSEFDRDAAMQRKLEKMADQAMTQMYKQARSEDKRAKVTSAMQTMLFTMLRKLDNDALNNIINNARDGCVPLNIIPLTTAAKLMVVIPDYNTYKNTCDGTTFTYASALWEIQQVVDADSKIVQLSEISMDNSPNLAWPLIVTALRANSAVKLQNNELSPVALRQMSCAAGTTQTACTDDNALAYYNTTKGGRFVLALLSDLQDLKWARFPKSDGTGTIYTELEPPCRFVTDTPKGPKVKYLYFIKGLNNLNRGMVLGSLAATVRLQAGNATEVPANSTVLSFCAFAVDAAKAYKDYLASGGQPITNCVKMLCTHTGTGQAITVTPEANMDQESFGGASCCLYCRCHIDHPNPKGFCDLKGKYVQIPTTCANDPVGFTLKNTVCTVCGMWKGYGCSCDQLREPMLQSADAQSFLNRVCGVSAARLTPCGTGTSTDVVYRAFDIYNDKVAGFAKFLKTNCCRFQEKDEDDNLIDSYFVVKRHTFSNYQHEETIYNLLKDCPAVAKHDFFKFRIDGDMVPHISRQRLTKYTMADLVYALRHFDEGNCDTLKEILVTYNCCDDDYFNKKDWYDFVENPDILRVYANLGERVRQALLKTVQFCDAMRNAGIVGVLTLDNQDLNGNWYDFGDFIQTTPGSGVPVVDSYYSLLMPILTLTRALTAESHVDTDLTKPYIKWDLLKYDFTEERLKLFDRYFKYWDQTYHPNCVNCLDDRCILHCANFNVLFSTVFPPTSFGPLVRKIFVDGVPFVVSTGYHFRELGVVHNQDVNLHSSRLSFKELLVYAADPAMHAASGNLLLDKRTTCFSVAALTNNVAFQTVKPGNFNKDFYDFAVSKGFFKEGSSVELKHFFFAQDGNAAISDYDYYRYNLPTMCDIRQLLFVVEVVDKYFDCYDGGCINANQVIVNNLDKSAGFPFNKWGKARLYYDSMSYEDQDALFAYTKRNVIPTITQMNLKYAISAKNRARTVAGVSICSTMTNRQFHQKLLKSIAATRGATVVIGTSKFYGGWHNMLKTVYSDVENPHLMGWDYPKCDRAMPNMLRIMASLVLARKHTTCCSLSHRFYRLANECAQVLSEMVMCGGSLYVKPGGTSSGDATTAYANSVFNICQAVTANVNALLSTDGNKIADKYVRNLQHRLYECLYRNRDVDTDFVNEFYAYLRKHFSMMILSDDAVVCFNSTYASQGLVASIKNFKSVLYYQNNVFMSEAKCWTETDLTKGPHEFCSQHTMLVKQGDDYVYLPYPDPSRILGAGCFVDDIVKTDGTLMIERFVSLAIDAYPLTKHPNQEYADVFHLYLQYIRKLHDELTGHMLDMYSVMLTNDNTSRYWEPEFYEAMYTPHTVLQAVGACVLCNSQTSLRCGACIRRPFLCCKCCYDHVISTSHKLVLSVNPYVCNAPGCDVTDVTQLYLGGMSYYCKSHKPPISFPLCANGQVFGLYKNTCVGSDNVTDFNAIATCDWTNAGDYILANTCTERLKLFAAETLKATEETFKLSYGIATVREVLSDRELHLSWEVGKPRPPLNRNYVFTGYRVTKNSKVQIGEYTFEKGDYGDAVVYRGTTTYKLNVGDYFVLTSHTVMPLSAPTLVPQEHYVRITGLYPTLNISDEFSSNVANYQKVGMQKYSTLQGPPGTGKSHFAIGLALYYPSARIVYTACSHAAVDALCEKALKYLPIDKCSRIIPARARVECFDKFKVNSTLEQYVFCTVNALPETTADIVVFDEISMATNYDLSVVNARLRAKHYVYIGDPAQLPAPRTLLTKGTLEPEYFNSVCRLMKTIGPDMFLGTCRRCPAEIVDTVSALVYDNKLKAHKDKSAQCFKMFYKGVITHDVSSAINRPQIGVVREFLTRNPAWRKAVFISPYNSQNAVASKILGLPTQTVDSSQGSEYDYVIFTQTTETAHSCNVNRFNVAITRAKVGILCIMSDRDLYDKLQFTSLEIPRRNVATLQAENVTGLFKDCSKVITGLHPTQAPTHLSVDTKFKTEGLCVDIPGIPKDMTYRRLISMMGFKMNYQVNGYPNMFITREEAIRHVRAWIGFDVEGCHATREAVGTNLPLQLGFSTGVNLVAVPTGYVDTPNNTDFSRVSAKPPPGDQFKHLIPLMYKGLPWNVVRIKIVQMLSDTLKNLSDRVVFVLWAHGFELTSMKYFVKIGPERTCCLCDRRATCFSTASDTYACWHHSIGFDYVYNPFMIDVQQWGFTGNLQSNHDLYCQVHGNAHVASCDAIMTRCLAVHECFVKRVDWTIEYPIIGDELKINAACRKVQHMVVKAALLADKFPVLHDIGNPKAIKCVPQADVEWKFYDAQPCSDKAYKIEELFYSYATHSDKFTDGVCLFWNCNVDRYPANSIVCRFDTRVLSNLNLPGCDGGSLYVNKHAFHTPAFDKSAFVNLKQLPFFYYSDSPCESHGKQVVSDIDYVPLKSATCITRCNLGGAVCRHHANEYRLYLDAYNMMISAGFSLWVYKQFDTYNLWNTFTRLQSLENVAFNVVNKGHFDGQQGEVPVSIINNTVYTKVDGVDVELFENKTTLPVNVAFELWAKRNIKPVPEVKILNNLGVDIAANTVIWDYKRDAPAHISTIGVCSMTDIAKKPTETICAPLTVFFDGRVDGQVDLFRNARNGVLITEGSVKGLQPSVGPKQASLNGVTLIGEAVKTQFNYYKKVDGVVQQLPETYFTQSRNLQEFKPRSQMEIDFLELAMDEFIERYKLEGYAFEHIVYGDFSHSQLGGLHLLIGLAKRFKESPFELEDFIPMDSTVKNYFITDAQTGSSKCVCSVIDLLLDDFVEIIKSQDLSVVSKVVKVTIDYTEISFMLWCKDGHVETFYPKLQSSQAWQPGVAMPNLYKMQRMLLEKCDLQNYGDSATLPKGIMMNVAKYTQLCQYLNTLTLAVPYNMRVIHFGAGSDKGVAPGTAVLRQWLPTGTLLVDSDLNDFVSDADSTLIGDCATVHTANKWDLIISDMYDPKTKNVTKENDSKEGFFTYICGFIQQKLALGGSVAIKITEHSWNADLYKLMGHFAWWTAFVTNVNASSSEAFLIGCNYLGKPREQIDGYVMHANYIFWRNTNPIQLSSYSLFDMSKFPLKLRGTAVMSLKEGQINDMILSLLSKGRLIIRENNRVVISSDVLVNN

SEQ ID NO: 3 - protein, 1246 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQ

SEQ ID NO: 4 - protein, 15 aa, Coronavirus
  source 1..15: note = Cornoavirus 2 isolate 2019-nCoV_HKU-SZ-005b_2020
MFVFLVLLPLVSSQC

SEQ ID NO: 5 - protein, 4 aa, Coronavirus
  source 1..4: note = Coronavirus 2 isolate 2019-nCoV_HKU-SZ-005b_2020
GSAS

SEQ ID NO: 6 - protein, 23 aa, synthetic construct
PTESIVRFPNITNLCPFGEVFNA

SEQ ID NO: 7 - protein, 23 aa, synthetic construct
YLYRLFRKSNLKPFERDISTEIY

SEQ ID NO: 8 - protein, 23 aa, synthetic construct
YRYRLFRKSNLKPFERDISKEIY